SEQUENCE LISTING (92 sequences)

SEQ ID NO: 1 - protein, 441 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 113 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSA

SEQ ID NO: 3 - protein, 7 aa, Artificial Sequence
  Description of Artificial Sequence Synthetic peptide
GFTFSRY

SEQ ID NO: 4 - protein, 4 aa, Artificial Sequence
  Description of Artificial Sequence Synthetic peptide
NSVG

SEQ ID NO: 5 - protein, 3 aa, Artificial Sequence
  Description of Artificial Sequence Synthetic peptide
GDY

SEQ ID NO: 6 - protein, 219 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 113 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKR

SEQ ID NO: 8 - protein, 13 aa, Artificial Sequence
  Description of Artificial Sequence Synthetic peptide
QSLIYSDGNAYLH

SEQ ID NO: 9 - protein, 6 aa, Artificial Sequence
  Description of Artificial Sequence Synthetic peptide
KVSNRF

SEQ ID NO: 10 - protein, 8 aa, Artificial Sequence
  Description of Artificial Sequence Synthetic peptide
QSTHVPWT

SEQ ID NO: 11 - protein, 453 aa, Artificial Sequence
  Description of Artificial Sequence Synthetic polypeptide
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 125 aa, Artificial Sequence
  Description of Artificial Sequence Synthetic polypeptide
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSA

SEQ ID NO: 13 - protein, 7 aa, Artificial Sequence
  Description of Artificial Sequence Synthetic peptide
GFAFSSY

SEQ ID NO: 14 - protein, 6 aa, Artificial Sequence
  Description of Artificial Sequence Synthetic peptide
WFDGTK

SEQ ID NO: 15 - protein, 15 aa, Artificial Sequence
  Description of Artificial Sequence Synthetic peptide
DRGIGARRGPYYMDV

SEQ ID NO: 16 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 18 - protein, 8 aa, Artificial Sequence
  Description of Artificial Sequence Synthetic peptide
QSISSYLN

SEQ ID NO: 19 - protein, 7 aa, Artificial Sequence
  Description of Artificial Sequence Synthetic peptide
AASSLQS

SEQ ID NO: 20 - protein, 9 aa, Artificial Sequence
  Description of Artificial Sequence Synthetic peptide
QQSYSTPLT

SEQ ID NO: 21 - protein, 440 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 114 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSA

SEQ ID NO: 23 - protein, 7 aa, Artificial Sequence
  Description of Artificial Sequence Synthetic peptide
GITFSNS

SEQ ID NO: 24 - protein, 6 aa, Artificial Sequence
  Description of Artificial Sequence Synthetic peptide
WYDGSK

SEQ ID NO: 25 - protein, 4 aa, Artificial Sequence
  Description of Artificial Sequence Synthetic peptide
NDDY

SEQ ID NO: 26 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 108 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 28 - protein, 8 aa, Artificial Sequence
  Description of Artificial Sequence Synthetic peptide
QSVSSYLA

SEQ ID NO: 29 - protein, 7 aa, Artificial Sequence
  Description of Artificial Sequence Synthetic peptide
DASNRAT

SEQ ID NO: 30 - protein, 9 aa, Artificial Sequence
  Description of Artificial Sequence Synthetic peptide
QQSSNWPRT

SEQ ID NO: 31 - protein, 231 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 32 - protein, 124 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSA

SEQ ID NO: 33 - protein, 7 aa, Artificial Sequence
  Description of Artificial Sequence Synthetic peptide
GYTFTNY

SEQ ID NO: 34 - protein, 6 aa, Artificial Sequence
  Description of Artificial Sequence Synthetic peptide
NTYTGE

SEQ ID NO: 35 - protein, 14 aa, Artificial Sequence
  Description of Artificial Sequence Synthetic peptide
YPHYYGSSHWYFDV

SEQ ID NO: 36 - protein, 214 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 108 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 38 - protein, 8 aa, Artificial Sequence
  Description of Artificial Sequence Synthetic peptide
QDISNYLN

SEQ ID NO: 39 - protein, 7 aa, Artificial Sequence
  Description of Artificial Sequence Synthetic peptide
FTSSLHS

SEQ ID NO: 40 - protein, 9 aa, Artificial Sequence
  Description of Artificial Sequence Synthetic peptide
QQYSTVPWT

SEQ ID NO: 41 - protein, 450 aa, Artificial Sequence
  Description of Artificial Sequence Synthetic polypeptide
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELLGGPSVFLFPPKPKDTLMISRTPEVTCWVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 123 aa, Artificial Sequence
  Description of Artificial Sequence Synthetic polypeptide
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSA

SEQ ID NO: 43 - protein, 7 aa, Artificial Sequence
  Description of Artificial Sequence Synthetic peptide
GYTFTSY

SEQ ID NO: 44 - protein, 6 aa, Artificial Sequence
  Description of Artificial Sequence Synthetic peptide
YPGNGD

SEQ ID NO: 45 - protein, 13 aa, Artificial Sequence
  Description of Artificial Sequence Synthetic peptide
VVYYSNSYWYFDV

SEQ ID NO: 46 - protein, 212 aa, Artificial Sequence
  Description of Artificial Sequence Synthetic polypeptide
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 107 aa, Artificial Sequence
  Description of Artificial Sequence Synthetic polypeptide
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 48 - protein, 7 aa, Artificial Sequence
  Description of Artificial Sequence Synthetic peptide
SSVSYMH

SEQ ID NO: 49 - protein, 7 aa, Artificial Sequence
  Description of Artificial Sequence Synthetic peptide
APSNLAS

SEQ ID NO: 50 - protein, 9 aa, Artificial Sequence
  Description of Artificial Sequence Synthetic peptide
QQWSFNPPT

SEQ ID NO: 51 - protein, 120 aa, Artificial Sequence
  Description of Artificial Sequence Synthetic polypeptide
VKLQQSGAELVKPGASVKLFCTASGFNIKDTYMHWVKQRPQQGLEWIGRIDPASGDTKYDPKFQVKATITADTSSNTAWLQLSSLTSEDTAVYYCADGMWVSTGYALDFWGQGTTVTVSS

SEQ ID NO: 52 - protein, 7 aa, Artificial Sequence
  Description of Artificial Sequence Synthetic peptide
GFNIKDT

SEQ ID NO: 53 - protein, 6 aa, Artificial Sequence
  Description of Artificial Sequence Synthetic peptide
DPASGD

SEQ ID NO: 54 - protein, 12 aa, Artificial Sequence
  Description of Artificial Sequence Synthetic peptide
GMWVSTGYALDF

SEQ ID NO: 55 - protein, 106 aa, Artificial Sequence
  Description of Artificial Sequence Synthetic polypeptide
SIVMTQTPKFLLVSAGDRVTITCKASQSVTNDVAWYQQKPGQSPKLLIYYASNRYTGVPDRFTGSGYGTDFTFTISTVQAEDLAVYFCQQDYSSPYTFGGGTKLEI

SEQ ID NO: 56 - protein, 8 aa, Artificial Sequence
  Description of Artificial Sequence Synthetic peptide
QSVTNDVA

SEQ ID NO: 57 - protein, 7 aa, Artificial Sequence
  Description of Artificial Sequence Synthetic peptide
YASNRYT

SEQ ID NO: 58 - protein, 9 aa, Artificial Sequence
  Description of Artificial Sequence Synthetic peptide
QQDYSSPYT

SEQ ID NO: 59 - protein, 443 aa, Artificial Sequence
  Description of Artificial Sequence Synthetic polypeptide
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 114 aa, Artificial Sequence
  Description of Artificial Sequence Synthetic polypeptide
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSA

SEQ ID NO: 61 - protein, 7 aa, Artificial Sequence
  Description of Artificial Sequence Synthetic peptide
GSSFTGY

SEQ ID NO: 62 - protein, 6 aa, Artificial Sequence
  Description of Artificial Sequence Synthetic peptide
DPYYGG

SEQ ID NO: 63 - protein, 4 aa, Artificial Sequence
  Description of Artificial Sequence Synthetic peptide
GMEY

SEQ ID NO: 64 - protein, 220 aa, Artificial Sequence
  Description of Artificial Sequence Synthetic polypeptide
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 114 aa, Artificial Sequence
  Description of Artificial Sequence Synthetic polypeptide
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKR

SEQ ID NO: 66 - protein, 13 aa, Artificial Sequence
  Description of Artificial Sequence Synthetic peptide
QSLVHRNGNTYLH

SEQ ID NO: 67 - protein, 7 aa, Artificial Sequence
  Description of Artificial Sequence Synthetic peptide
KVSNRFS

SEQ ID NO: 68 - protein, 10 aa, Artificial Sequence
  Description of Artificial Sequence Synthetic peptide
SQSTHVPPLT

SEQ ID NO: 69 - protein, 456 aa, Artificial Sequence
  Description of Artificial Sequence Synthetic polypeptide
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 127 aa, Artificial Sequence
  Description of Artificial Sequence Synthetic polypeptide
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSA

SEQ ID NO: 71 - protein, 7 aa, Artificial Sequence
  Description of Artificial Sequence Synthetic peptide
GFTFSSY

SEQ ID NO: 72 - protein, 6 aa, Artificial Sequence
  Description of Artificial Sequence Synthetic peptide
NASGTR

SEQ ID NO: 73 - protein, 17 aa, Artificial Sequence
  Description of Artificial Sequence Synthetic peptide
GKGNTHKPYGYVRYFDV

SEQ ID NO: 74 - protein, 215 aa, Artificial Sequence
  Description of Artificial Sequence Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 109 aa, Artificial Sequence
  Description of Artificial Sequence Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKR

SEQ ID NO: 76 - protein, 9 aa, Artificial Sequence
  Description of Artificial Sequence Synthetic peptide
QSVSSSYLA

SEQ ID NO: 77 - protein, 7 aa, Artificial Sequence
  Description of Artificial Sequence Synthetic peptide
GASSRAT

SEQ ID NO: 78 - protein, 9 aa, Artificial Sequence
  Description of Artificial Sequence Synthetic peptide
LQIYNMPIT

SEQ ID NO: 79 - protein, 454 aa, Artificial Sequence
  Description of Artificial Sequence Synthetic polypeptide
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 125 aa, Artificial Sequence
  Description of Artificial Sequence Synthetic polypeptide
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSA

SEQ ID NO: 81 - protein, 219 aa, Artificial Sequence
  Description of Artificial Sequence Synthetic polypeptide
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 113 aa, Artificial Sequence
  Description of Artificial Sequence Synthetic polypeptide
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKR

SEQ ID NO: 83 - protein, 448 aa, Artificial Sequence
  Description of Artificial Sequence Synthetic polypeptide
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 119 aa, Artificial Sequence
  Description of Artificial Sequence Synthetic polypeptide
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSA

SEQ ID NO: 85 - protein, 7 aa, Artificial Sequence
  Description of Artificial Sequence Synthetic peptide
GYTFTSY

SEQ ID NO: 86 - protein, 6 aa, Artificial Sequence
  Description of Artificial Sequence Synthetic peptide
YPGNGD

SEQ ID NO: 87 - protein, 9 aa, Artificial Sequence
  Description of Artificial Sequence Synthetic peptide
YDYNYAMDY

SEQ ID NO: 88 - protein, 213 aa, Artificial Sequence
  Description of Artificial Sequence Synthetic polypeptide
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 107 aa, Artificial Sequence
  Description of Artificial Sequence Synthetic polypeptide
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKR

SEQ ID NO: 90 - protein, 7 aa, Artificial Sequence
  Description of Artificial Sequence Synthetic peptide
SSVSYMH

SEQ ID NO: 91 - protein, 7 aa, Artificial Sequence
  Description of Artificial Sequence Synthetic peptide
ATSNLAS

SEQ ID NO: 92 - protein, 9 aa, Artificial Sequence
  Description of Artificial Sequence Synthetic peptide
QQWTFNPPT

(19) United States
(12) Patent Application Publication
(10) Pub. No.: US 2022/0409738 A1
(43) Pub. Date: Dec. 29, 2022

(54) EXTRACELLULAR VESICLES AND USES THEREOF FOR ANTIBODY DELIVERY

(71) Applicant: Aruna Bio, Inc., Athens, GA (US)

(72) Inventors: Steven L. Stice, Athens, GA (US); Raymond Swetenburg, Athens, GA (US); Rhianna K. Carty, Athens, GA (US)

(73) Assignee: Aruna Bio, Inc., Athens, GA (US)

(21) Appl. No.: 17/735,971

(22) Filed: May 3, 2022

Related U.S. Application Data
(63) Continuation of application No. PCT/US2020/058968, filed on Nov. 4, 2020.
(60) Provisional application No. 62/930,178, filed on Nov. 4, 2019.

(51) Int. Cl. A61K 47/69; C07K 16/00; A61K 35/30; A61K 9/127

(52) U.S. Cl. CPC A61K 47/6901; C07K 16/00; A61K 35/30; A61K 9/127; A61K 2039/505

(57) ABSTRACT

Disclosed herein are methods of delivering a polypeptide, e.g., an antibody or antigen binding portion thereof, to the central nervous system of a subject, by administering to the subject the polypeptide (e.g., antibody or antigen-binding portion thereof) conjugated to the surface of an extracellular vesicle (EV) derived from a neural cell, e.g., a neural progenitor cell or a neural stem cell. Conjugates comprising neural EVs coupled to a polypeptide, such as an antibody or antigen binding portion thereof, and methods of use thereof, are also provided. Also disclosed herein are methods of delivering a polypeptide, e.g., an antibody or antigen binding portion thereof, by administering to the subject the polypeptide (e.g., antibody or antigen-binding portion thereof) loaded within the lumen of an extracellular vesicle (EV) derived from neural cells.

### RELATED APPLICATIONS

This application is a continuation of International Application No. PCT/US2020/058968 filed Nov. 4, 2020 which claims priority to U.S. Provisional Application No. 62/930,178, filed Nov. 4, 2019, the entire contents of each of which is incorporated herein by reference for all purposes.

### SEQUENCE LISTING

The instant application contains a Sequence Listing which has been submitted electronically in ASCII format, and which is hereby incorporated by reference in its entirety. Said ASCII copy, created on May 3, 2022, is named A106525_1040_US.C1_SL.txt, and is 86,435 bytes in size.

### BACKGROUND

Recent failures of antibody-based drugs, including the Alzheimer's disease treatments Solanezumab and Aducanumab, have raised a number of questions about the future of therapeutic antibody treatments in the CNS, including the use of IV as a route of administration for effective bioavailability in the CNS. In mice, antibody concentrations are measurable in the brain at about 0.1% of the injected dose when given by IV, and detectable up to 72 hours later, although in very low quantities (Banks et al., Peptides, 2002. 23(12): p. 2223-6). Similarly, IV-delivered antibodies have reported CSF levels of 0.1% of the concentration found in the serum in human clinical trials, indicating that IgG does not readily cross the human blood-brain barrier (BBB) (Rubenstein et al., Blood. 2003. 101(2): p. 466-8).

Bispecific antibodies have been generated which contain one variable region binding a BBB transcytosis protein, while the other variable region binds to a specific target of interest. Notably, the transferrin receptor has been shown to be a potent transporter across the BBB, but its use in human trials has been hindered by safety concerns (Couch et al., Sci Transl Med, 2013. 5(183): p. 183ra57, 1-12). Recently, other potential bispecific targets have been explored, including the BBB-traversing CD98hc (Zuchero et al., Neuron, 2016. 89(1): p. 70-82) and FC5 (Muruganandam et al., 2002. 16(2): p. 240-242), but none have been proven in the clinic as yet. Adeno-associated viruses (AAV) have also shown promise both in their ability to cross the BBB (Deverman et al., Nat Biotechnol, 2016. 34(2): p. 204-9. and deliver exogenous genes which can code for secreted antibodies in the brain (Ryan et al., 2010. 18(8): p. 1471-1481). However, AAV are prone to high clearance rates, particularly upon re-administration, as well as the potential downfalls of a neuron or other CNS cell devoting too much energy to generating these antibodies. There is therefore a need in the art for additional methods of delivering therapeutic proteins, such as antibodies, to the central nervous system.

### SUMMARY OF THE INVENTION

To improve the delivery of therapeutic proteins, such as antibodies, to the brain and central nervous system of a subject, provided herein are methods of using neural extracellular vesicles (EVs) as carriers for therapeutic proteins. In one aspect, provided herein is a method of delivering an antibody, or antigen binding portion thereof, to the central nervous system of a subject, comprising administering to the subject a conjugate comprising the antibody or antigen binding portion thereof and an extracellular vesicle derived from a neural cell, wherein the antibody or antigen binding portion thereof is conjugated to the surface of the extracellular vesicle and associated proteins by way of a click linker.

In one aspect, provided herein is a method of delivering a polypeptide to the central nervous system of a subject, comprising administering to the subject a conjugate comprising the polypeptide and an extracellular vesicle derived from a neural cell, wherein the polypeptide is conjugated to the surface of the extracellular vesicle and associated proteins by way of a click linker. In exemplary embodiments, the polypeptide is an exogenous polypeptide. In other exemplary embodiments, the polypeptide is a therapeutic polypeptide.

In some embodiments, the conjugate is administered intravenously.

In some embodiments, the conjugate is administered intranasally.

In some embodiments, the conjugate is delivered to the brain of the subject. In some embodiments, the conjugate is delivered across the blood brain barrier of the subject.

In some embodiments, the neural cell is a neural progenitor cell. In some such embodiments, the neural progenitor cell is derived from a human pluripotent cell. In certain embodiments, the human pluripotent cell is a human embryonic stem cell. In other embodiments, the human pluripotent cell is an induced pluripotent stem cell.

In some embodiments, the antibody, or antigen-binding portion thereof, is an IgG.

In some embodiments, the antibody, or antigen-binding portion thereof, is an antibody fragment selected from the group consisting of a Fab, a F(ab′)2, an scFv, a tandem scFv, a diabody, a minibody, and a single domain antibody.

In some embodiments, the antibody or antigen binding portion thereof is a humanized antibody or antigen binding portion thereof.

In some embodiments, the antibody or antigen binding portion thereof is a fully human antibody or antigen binding portion thereof.

In some embodiments, the click linker is formed from reaction between an azide click reagent and an alkyne click reagent.

In some embodiments, the click linker is formed from reaction between azide and dibenzocyclooctyne (DBCO).

In some embodiments, the click linker is formed from reaction between tetrazine and transcyclooctene.

In some embodiments, the click linker is formed from reaction between tetrazine and norbornene.

In some embodiments, the antibody is delivered to the brain of the subject.

In some embodiments, the antibody is delivered to the central nervous system of the subject.

In some embodiments, the EV further comprises an exogenous nucleic acid and/or an exogenous protein. In certain embodiments, the EV comprises a siRNA and/or an antisense nucleic acid.

In some aspects, provided here is a composition comprising an antibody-EV (Ab-EV) conjugate, the conjugate comprising an antibody, or antigen-binding portion thereof, and an extracellular vesicle (EV) derived from a neural cell, wherein the antibody, or antigen-binding portion thereof, is conjugated to the EV surface by a click linker.

In some embodiments, the neural cell is a neural progenitor cell. In some such embodiments, the neural progenitor cell is derived from a human pluripotent cell. In certain embodiments, the human pluripotent cell is a human embryonic stem cell. In other embodiments, the human pluripotent cell is an induced pluripotent stem cell.

In some embodiments, the click linker is formed between the reaction of any one or more of: azide and dibenzocyclooctyne; tetrazine and transcyclooctene; tetrazein and norbornene; azide and alyne; azide (strain-promoted) and alkyne; azide (strain-promoted) and nitrone; alkene and azide; alkene and tetrazine; and/or alkene and tetrazole.

In some embodiments, the antibody, or antigen-binding portion thereof, is an IgG.

In some embodiments, the antibody, or antigen-binding portion thereof, is an antibody fragment selected from the group consisting of a Fab, a F(ab′)2, an scFv, a tandem scFv, a diabody, a minibody, and a single domain antibody.

In some embodiments, the antibody, or antigen binding portion thereof is a humanized antibody or antigen binding portion thereof.

In some embodiments, the antibody, or antigen-binding portion thereof, is any one of more of solanezumab, aducanumab, nivolumab, bevacizumab, ocrelizumab, natalizumab, dinutuximab, gantenerumab, lecanemab, or ublituximab.

In some aspects, provided herein is a method of loading an antibody, or antigen-binding portion thereof, into the lumen of an extracellular vesicle (EV), comprising: i) treating the EV with saponin to permeabilize the EV membrane; ii) sonicating the treated EV; and iii) adding the antibody, or antigen-binding portion thereof, to the EV, thereby loading an antibody, or antigen-binding portion thereof, into the lumen of the EV.

In some embodiments, the EV is treated with about 0.05% to 0.3% saponin.

In some embodiments, the method of loading an antibody, or antigen-binding portion thereof, into the lumen of an extracellular vesicle (EV) comprises incubating the EV upon loading the antibody, or antigen-binding portion thereof for a period of time sufficient for at least 10% of the antibody to be loaded into the EVs.

In some aspects, provided herein is a method of delivering an antibody, or antigen binding portion thereof, to the central nervous system (CNS) of a subject, comprising administering to the subject an extracellular vesicle (EV) that comprises the antibody, or antigen binding-portion thereof, in the lumen of the EV, wherein the EV is derived from a neural cell.

In some embodiments, the neural cell is a neural progenitor cell. In some such embodiments, the neural progenitor cell is derived from a human pluripotent cell. In certain embodiments, the human pluripotent cell is a human embryonic stem cell. In other embodiments, the human pluripotent cell is an induced pluripotent stem cell.

In some embodiments, the antibody, or antigen-binding portion thereof, is an IgG.

In some embodiments, the antibody, or antigen-binding portion thereof, is an antibody fragment selected from the group consisting of a Fab, a F(ab′)₂, an scFv, a tandem scFv, a diabody, a minibody, and a single domain antibody.

In some embodiments, the antibody or antigen binding portion thereof is a humanized antibody or antigen binding portion thereof.

In some embodiments, the antibody or antigen binding portion thereof is a fully human antibody or antigen binding portion thereof.

In some embodiments, the antibody is delivered to the brain of the subject.

In some embodiments, the antibody is delivered to the spinal cord of the subject.

In some embodiments, the EV further comprises an exogenous nucleic acid and/or an exogenous protein. In some embodiments, the EV comprises an exogenous siRNA and/or an antisense nucleic acid. In some embodiments, the EV further comprises a small molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic depicting an exemplary method of conjugating an antibody (Ab) to an extracellular vesicle (EV) using a copperless click chemistry reaction. Azido groups were added to a non-reactive antibody. Separately, extracellular vesicles (EV) were modified with DIBO groups via SDP coupling which attacks amine groups. A copperless click chemistry reaction then conjugated the antibody to EV when mixed.

FIG. 2A and FIG. 2B provide images of mouse brain sections obtained from mice administered an unconjugated antibody (FIG. 2A) or an antibody conjugated to extracellular vesicles derived from neural progenitor cells (FIG. 2B). Nuclei (blue) and antibody (white) signal is shown.

FIG. 3A and FIG. 3B illustrate loading efficiency of an antibody (FIG. 3A) or luciferase protein (FIG. 3B) into the lumen of extracellular vesicles derived from neural progenitor cells (AB126) using various loading conditions.

### DETAILED DESCRIPTION OF THE INVENTION

### A. Definitions

In order that the invention may be more readily understood, certain terms are first defined. In addition, it should be noted that whenever a value or range of values of a parameter are recited, it is intended that values and ranges intermediate to the recited values are also part of this invention.

The term “about” or “approximately” usually means within 5%, or more preferably within 1%, of a given value or range.

The term “antibody” is used herein in the broadest sense and encompasses various structures that bind a target antigen, including but not limited to monoclonal antibodies, polyclonal antibodies, multispecific antibodies (e.g., bispecific antibodies), nanobodies, monobodies, antibody mimetics, and antibody fragments that exhibit the desired antigen-binding activity.

In some embodiments, an antibody includes an immunoglobulin molecule comprising four polypeptide chains—two heavy (H) chains and two light (L) chains inter-connected by disulfide bonds—as well as multimers thereof (e.g., IgM). Each heavy chain (HC) comprises a heavy chain variable region (or domain) (abbreviated herein as HCVR or VH) and a heavy chain constant region (or domain). The heavy chain constant region comprises three domains, CH1, CH2 and CH3. Each light chain (LC) comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other.

The term “Fc domain” is used to define the C-terminal region of an immunoglobulin heavy chain, which may be generated by papain digestion of an intact antibody. The Fc domain may be a native sequence Fc domain or a variant Fc domain. The Fc domain of an immunoglobulin generally comprises two constant domains, a CH2 domain and a CH3 domain, and optionally can comprise a CH4 domain. Replacements of amino acid residues in the Fc portion to alter antibody effector function are known in the art (Winter, et al. U.S. Pat. Nos. 5,648,260; 5,624,821). The Fc domain of an antibody mediates several important effector functions, e.g., cytokine induction, ADCC, phagocytosis, complement dependent cytotoxicity (CDC) and half-life/clearance rate of antibody and antigen-antibody complexes. In certain embodiments, at least one amino acid residue is altered (e.g., deleted, inserted, or replaced) in the Fc domain of an Fc domain-containing binding protein such that effector functions of the binding protein are altered.

An “intact” or a “full length” antibody, as used herein, refers to an antibody comprising four polypeptide chains, two heavy (H) chains and two light (L) chains. In one embodiment, an intact antibody is an intact IgG antibody.

The term “monoclonal antibody” as used herein refers to an antibody obtained from a population of substantially homogeneous antibodies, i.e., the individual antibodies comprising the population are identical in sequence and bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “human antibody”, as used herein, refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “humanized antibody” is intended to refer to an antibody in which CDR sequences derived from the germline of one non-human mammalian species, such as a mouse, have been grafted onto human framework sequences. Additional framework region modifications may be made within the human framework sequences and/or non-human CDR sequences. A “humanized form” of an antibody, e.g., a non-human antibody, refers to an antibody that has undergone humanization.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

An “antibody fragment”, “antigen-binding fragment” or “antigen-binding portion” of an antibody refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)2, diabodies, linear antibodies, single-chain antibody molecules (e.g. scFv), and multispecific antibodies formed from antibody fragments.

A “multispecific antigen binding polypeptide” or “multispecific antibody” is one that targets more than one antigen or epitope. A “bispecific,” “dual-specific” or “bifunctional” antigen binding polypeptide or antibody is a hybrid molecule comprising two different antigen binding sites. Bispecific antigen binding polypeptides and antibodies are examples of multispecific antigen binding polypeptides or multispecific antibodies, and may be produced by a variety of methods including, but not limited to, fusion of hybridomas or linking of Fab′ fragments or half-antibodies. See, e.g., Songsivilai and Lachmann, 1990, Clin. Exp. Immunol. 79:315-321; Kostelny et al., 1992, J. Immunol. 148:1547-1553, Brinkmann and Kontermann. 2017. MABS. 9(2):182-212. The two binding sites of a bispecific antigen binding polypeptide or antibody, for example, will bind to two different epitopes, which may reside on the same or different protein targets.

The term “antibody mimetic” or “antibody mimic” refers to a molecule that is not structurally related to an antibody but is capable of specifically binding to an antigen. Examples of antibody mimetics include, but are not limited to, an adnectin (i.e., fibronectin based binding molecules), an affilin, an affimer, an affitin, an alphabody, an affibody, DARPins, an anticalin, an avimer, a fynomer, a Kunitz domain peptide, a monobody, a nanoCLAMP, a nanobody, a unibody, a versabody, an aptamer, and a peptidic molecule all of which employ binding structures that, while they mimic traditional antibody binding, are generated from and function via distinct mechanisms. The term “autologous EV” is used to describe a population of EVs which are obtained from cells from a subject or patient to whom the EVs are to be administered.

As used herein, the term “central nervous system” or “CNS” refers to all structures within the dura matter. Such structures include, but are not limited to, the cells and tissue of the brain and spinal cord. The CNS also comprises the cerebrospinal fluid, which fills the ventricles of the brain and the central canal of the spinal cord.

As used herein, the term “effective amount” or “therapeutically effective amount” refers to the amount of an agent, e.g., a composition comprising EVs, e.g., exosomes, which is sufficient to reduce or ameliorate the severity and/or duration of a disorder, or one or more symptoms thereof, prevent the advancement of a disorder, cause regression of a disorder, prevent the recurrence, development, onset, or progression of one or more symptoms associated with a disorder, or enhance or improve the prophylactic or therapeutic effect(s) of another therapy.

The term “embryonic stem cell” refers to pluripotent cells, preferably of primates, including humans, which are isolated from the blastocyst stage embryo.

The terms “extracellular vesicle” and “EV” are used herein to refer to a vesicle of about 10 nm to 10 μm in size enclosed by a lipid bilayer, e.g., a portion of a plasma membrane. EVs can contain fluid, macro-molecules, solutes, and metabolites from a cell.

The term “EV” also includes lipid vesicles engineered to contain bioactive molecules found in a cell-derived EVs, such as neural EVs. These terms encompass both exosomes and ectosomes. EVs may be obtained from the appropriate biological source using a combination of isolation techniques, for example, centrifugation, filtration and ultracentrifugation methodologies. Exosomes are released on the exocytosis of multivesicular bodies (MVBs). Ectosomes are vesicles assembled at and released from the plasma membrane. In some cases, the EV is about 20 nm to 10 μm, 20 nm to 1 μm, 20 nm-500 nm, 20 nm-200 nm, 30 nm-100 nm, 30 nm-160 nm, or 80-160 nm in size. In some embodiments, the EVs are exosomes that are about 20 to 150 nm in size. EVs may be isolated from any suitable biological sample from a mammal, including but not limited to, whole blood, serum, plasma, breast milk, cerebrospinal fluid, amniotic fluid, ascitic fluid, or bone marrow. In some embodiments, EVs can be isolated from cultured mammalian cells (e.g. immature dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like). In exemplary embodiments, EVs can be isolated from a neural cell (e.g., a neural progenitor cell, a neural stem cell, a glial cell, an astrocyte, a neuron, etc.).

The term “human Pluripotent Stem Cells”, of which “human Embryonic Stem Cells” (hESCs) and human induced pluripotent stem cells (hiPSCs) are a subset, are derived from pre-embryonic, embryonic, fetal tissue or adult stem cells (in the case of human induced pluripotent stem cells) at any time after fertilization, and have the characteristic of being capable under appropriate conditions of producing progeny of several different cell types, especially including neuronal stem and progenitors, neural crest cells, mesenchymal stem cells (MSCs) and related proliferative and non-proliferative neural cells. The term includes both established lines of stem cells of various kinds, and cells obtained from primary tissue that are pluripotent in the manner described.

“Pharmaceutically acceptable” as used herein, refers to a material, such as a carrier, excipient or diluent, which does not abrogate the biological activity or properties of the compound, and is relatively nontoxic, i.e., the material may be administered to an individual without causing undesirable biological effects or interacting in a deleterious manner with any of the components of the composition in which it is contained. In some instances, a “pharmaceutically acceptable” carrier or excipient refers to those compounds that are suitable for use in contact with the tissues of human beings and animals without excessive toxicity, irritation, allergic response, immunogenicity, or complications commensurate with a reasonable benefit/risk ratio.

The term “linker” as used herein means a divalent chemical moiety comprising a covalent bond or a chain of atoms that covalently attaches an antibody to an EV to form an antibody-EV conjugate. Any known method of conjugation of peptides or macromolecules can be used in the context of the present disclosure. Generally, covalent attachment of the antibody and the EV requires the linker to have two reactive functional groups, i.e., bivalency in a reactive sense. Bivalent linker reagents which are useful to attach two or more functional or biologically active moieties are known, and methods for such conjugation have been described in, for example, Hermanson, G. T. (1996) Bioconjugate Techniques; Academic Press: New York, p 234-242, the disclosure of which is incorporated herein by reference as it pertains to linkers suitable for covalent conjugation. In some embodiments, the compositions and methods described herein make use of a “click linker,” which is generated from a reaction between two complementary click functional groups.

When the term “linker” is used in describing the linker in conjugated form, one or both of the reactive termini will be absent (having been converted to a chemical moiety) or incomplete (such as being only the carbonyl of a carboxylic acid) because of the formation of the bonds between the linker and the extracellular cell membrane binding moiety, and/or between the linker and the site-directed modifying polypeptide. Accordingly, linkers useful herein include, without limitation, linkers containing a chemical moiety formed by a coupling reaction between a reactive functional group on the linker and a nucleophilic group or otherwise reactive substituent on the antibody, and a chemical moiety formed by a coupling reaction between a reactive functional group on the linker and a nucleophilic group on the EV.

Examples of chemical moieties formed by these coupling reactions result from reactions between chemically reactive functional groups, including a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive functional groups to form the chemical moiety include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Suitable linkers may contain an electrophilic functional group for reaction with a nucleophilic functional group on the antibody, the EV, or both.

The term “neural cell” is a cell pertaining to a nerve or nerves, which are the cordlike bundles of fibers made up of neurons. A neural cell is typically derived from a neural progenitor cell (NPC). In some embodiments, neural cells can be derived in vitro from neural progenitor cells or from pluripotent stem cells. In exemplary embodiments, the neural cells referenced herein are human neural cells. In some embodiments, the neural cell can be a neuron, a glial cell, an astrocyte, an oligodendrocyte, or a microglial cell, a Schwann cell, or a glioma cell. In other embodiments, the neural cell can be a neural progenitor cell or a neural stem cell. The terms “neural progenitor cell” and “neural stem cell” refer to multipotent cells that have the capacity to differentiate into a restricted repertoire of neuronal and glial cell types. In some embodiments, neural progenitor cells can be derived in vitro from pluripotent stem cells, e.g., induced pluripotent stem cells (iPS cells) or embryonic stem cells (ES cells). In exemplary embodiments, the neural progenitor cells referenced herein are human neural progenitor cells. In some embodiments, the neural progenitor cells can be non-transformed. In some embodiments, the neural progenitor cells are proliferative. In some embodiments, the neural progenitor cells maintain phenotype without differentiation.

The term “neural EV” is used to refer to a cell-derived EV derived from neural cells, e.g., neural progenitor cells. The term also refers to vesicles engineered to contain a sufficient number of the bioactive molecules found in cell-derived neural EV to have substantially the same bioactivity.

“Neurovascular repair” described herein, refers to the recovery that occurs after a neurovascular injury to the brain. Neurovascular injury refers to damage to the major blood vessels supplying the brain, brainstem, and upper spinal cord, including the vertebral, basilar, and carotid arteries. These vessels are located both extra- and intracranially, and injuries can occur in either or both of these locations. In one instance, neurovascular repair occurs when there is an increase in neurogenesis and angiogenesis and/or the recruitment of glial cells to clear the hemorrhage and create an environment for healing. Astrocytes, pericytes, matrix proteases, and vascular smooth muscle cells also play significant roles in neurovascular repair.

As used herein, the term “sample” refers to a specimen (e.g., cell (e.g., neural cell), tissue, blood, blood component (e.g., serum or plasma), urine, saliva, amniotic fluid, cerebrospinal fluid, pancreatic fluid, chorionic villus sample, and bone marrow) taken from a subject.

As used herein, the term “subject” refers to any organism that is the target of administration or treatment. A “subject” can be an organism, for example, a mammal (e.g., a human, a non-human mammal, a non-human primate, a primate, a laboratory animal, a mouse, a rat, a hamster, a cat, or a dog). In one embodiment, a subject is a human subject. The term “patient” refers to a human subject under the treatment of a clinician, e.g., physician. A subject can be male or female.

The term “treat” and “treatment” refers to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. Treatment does not require the complete amelioration of a symptom or disease and encompasses embodiments in which one reduces symptoms and/or underlying risk factors. “Treatment” can also mean prolonging survival as compared to expected survival if not receiving treatment. Those in need of treatment include those already with the condition or disorder, as well as those prone to have the condition or disorder or those in which the condition or disorder is to be prevented. The term “prevent” does not require the 100% elimination of the possibility of an event. Rather, it denotes that the likelihood of the occurrence of the event has been reduced in the presence of the compound or method.

In accordance with the present invention there may be employed conventional cell culture methods, chemical synthetic methods and other biological and pharmaceutical techniques within the skill of the art. Such techniques are well-known and are otherwise explained fully in the literature. Standard techniques for growing cells, separating cells, and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is to be noted that as used herein and in the appended claims, the singular forms “a,” “and” and “the” include plural references unless the context clearly dictates otherwise.

### B. Extracellular Vesicles (EVs) and Delivery of Therapeutic Proteins

The present invention is directed to methods of delivering a therapeutic polypeptide, such as an antibody, to the central nervous system of a subject, e.g., across the blood brain barrier of a subject, by administering the polypeptide conjugated to the surface of neural extracellular vesicles (EVs), such as, for example, exosomes and/or microvesicles derived from neural cells. Further provided are methods of using polypeptide-EV conjugates, e.g., antibody (Ab)-EV conjugates, in various applications relating to the treatment of neurological disorders and injuries, including but not limited to Alzheimer's Disease, Huntington's Disease, Parkinson's Disease, dementia, neurological cancer, e.g., glioblastoma, or cancers that have metastasized to the central nervous system. Additionally, conjugates comprising neural EVs coupled to an antibody or antigen-binding fragment thereof, and methods of use thereof, are provided herein.

The present invention also relates to methods of delivering a therapeutic polypeptide, such as an antibody, to the central nervous system of a subject, e.g., across the blood brain barrier of a subject, by administering the polypeptide loaded within the lumen of neural extracellular vesicles (EVs), e.g., exosomes and/or microvesicles derived from neural cells. Further provided are methods of using luminal-loaded EVs in various applications relating to the treatment of neurological disorders and injuries, including but not limited to Alzheimer's Disease, Huntington's Disease, Parkinson's Disease, dementia, neurological cancer, e.g., glioblastoma, or cancers that have metastasized to the central nervous system.

Extracellular vesicles comprise a heterogeneous group of small structures enclosed by a lipid bilayer, for example, a portion of a cellular plasma membrane. EVs can range in size from about 10 nm to 10 μm in diameter, and most commonly fall within the range of about 25-500 nm. EVs can broadly be divided into two classes, exosomes and ectosomes. Exosomes can be formed by cells through the inward budding of the endosomal membrane during the maturation of multivesicular bodies (MVBs). Exosomes can then be released to the extracellular space by fusion of MVBs with the cell surface. Exosomes are typically 25-500 nm in diameter, and in some embodiments can be within the range of about 25-250 nm, about 50-150 nm, or about 50-200 nm. Ectosomes, also known as microvesicles, can be formed by cells through budding of the plasma membrane. Ectosomes can vary in size from about 10 nm to 10 μm, and in some embodiments can be within the range of about 10-1000 nm, or about 50-500 nm.

EVs suitable for use in the conjugates and methods of the invention can be derived from any suitable source. For example, EVs can be derived from neural cells, such as neural stem cells, neural progenitor cells, or differentiated neural cells, such as neurons, glial cells, or astrocytes. EVs suitable for use in the methods described herein can also be produced synthetically. In exemplary embodiments, EVs used in the antibody conjugates described herein are derived from neural progenitor cells or neural stem cells.

EVs are involved in intercellular communication, allowing for the transfer of material from EVs to cells by fusion with the cell membrane or via cellular uptake including but not limited to calveolin-independent and -dependent mechanisms, clathrin-independent and -dependent mechanisms, macropinocytosis and/or phagocytosis. EVs have been reported to be involved in numerous physiological processes, including immune modulation, angiogenesis, migration of endothelial cells in connection with tumor growth, or reducing damage in ischemia reperfusion injury. Many of these functions are mediated by proteins, nucleic acids, or lipids contained in or on the vesicles.

EVs can carry cargo in the lumen, and embedded in or attached to the lipid bilayer. This cargo can include proteins, lipids, and/or nucleic acids, for example, mRNA or miRNA. In cases where EVs are produced by cells, the composition of the cargo is highly dependent on cell type. For example, it has been shown that EVs derived from astrocytes, neural progenitor cells, and mesenchymal stem cells each contain a distinct complement of protein cargo (see U.S. Patent Application Publication No. US2018/0327714A1, the entire contents of which are incorporated herein by reference). EVs derived from these different cell types also contain a distinct profile of nucleic acid molecules, including mRNA and/or miRNA. In embodiments where EVs are obtained from cells, the EVs contain endogenous cargo that reflects the contents of EVs produced by the cells from which the EVs are derived. In some embodiments, EVs obtained from cells can also contain exogenous cargo. Exogenous cargo includes proteins, nucleic acids, small molecules, or lipids that are introduced into EVs by manipulation of the vesicles following their release into the extracellular space. In other embodiments, EVs obtained from cells can contain exogenous cargo, which is packaged into EVs as a result of a recombinant nucleic acid present in the cells from which the EVs are derived. For example, EVs containing a recombinant protein can be derived from cells that contain a recombinant nucleic acid encoding the protein. In embodiments where EVs are produced synthetically, suitable cargo can be selected for inclusion in the vesicles. For example, in one embodiment, synthetic EVs can contain one or more proteins, lipids, or nucleic acids present in EVs derived from neural cells, e.g., neural progenitor cells, neurons, glial cells, or astrocytes.

EVs, e.g., exosomes, derived from cells comprise a variety of biological molecules that reflect their cellular origin. In one embodiment, the invention provides a population of EVs derived from neural cells, e.g., neural progenitor cells. Such neural EVs contain a milieu of different proteins, including cytokines and growth factors, and coding and noncoding RNA molecules, derived from neural cells. The cargo contained in neural EVs can impact neural and vascular function by providing neuroprotection, reducing inflammation, immunomodulation via acting on T cells, macrophages and microglia, reducing oxidative stress, improving vascular integrity, impacting metabolic activity, and inducing a neuroregenerative effect via an increase in neurogenesis, cell migration, re-myelination and differentiation. Without wishing to be bound by theory, the combination of native proteins expressed on the surface of EVs derived from neural cells (e.g., neural progenitor cells, neural stem cells) is thought to allow the vesicles to localize to the CNS and traverse the blood brain barrier to a greater extent than EVs derived from other sources. The ability to target the CNS and/or cross the blood brain barrier is not impeded by surface labeling the EVs with antibodies, as shown herein. Moreover, EV-antibody conjugates comprising EVs derived from neural cells (e.g., neural progenitor cells, neural stem cells) are able to transport functionally intact antibodies, and antigen binding fragments thereof, across the blood brain barrier for delivery to targets in the brain and central nervous system.

In some embodiments, neural cell derived EVs, e.g., exosomes, can contain membrane proteins, including but not limited to CD63, CD81, and CD133. Accordingly, in one embodiment, the EVs, e.g., exosomes, suitable for use in the conjugates and methods described herein comprise one or more (i.e., one or more, two or more, or all three) of the following cell surface proteins: CD63, CD81, and CD133. In addition, the EVs, e.g., exosomes, can comprise one or more (i.e., one or more, two or more, three or more, four or more, five or more, six or more, seven or more, eight or more, nine or more, ten or more, eleven or more, or all twelve) of the following miRNAs: hsa-miR-135a-2, hsa-miR-124-1, hsa-miR-124-2, hsa-miR-124-3, hsa-miR-489, hsa-miR-9-3, hsa-miR-9-2, hsa-miR-9-1, hsa-miR-219b, hsa-miR-219a-2, hsa-miR-363 and/or hsa-miR-20b. In one embodiment, the isolated population of EVs are enriched for one or more of the foregoing miRNAs.

Enrichment can be measured in absolute or relative quantities, such as when compared to unmodified exosomes derived from non-neural cells, e.g., mesenchymal stem cells.

(i) EV Production

EVs suitable for use in the conjugates and methods disclosed herein can be produced by a variety of cell types, or can be produced synthetically. In one embodiment, EVs can be derived from neural cells, such as neural progenitor cells, neurons, astrocytes, oligodendrocytes, microglia, Schwann cells, or glioma cells. In some embodiments, EVs can be produced by transformed cell lines. In other embodiments, EVs can be produced by non-transformed cells. In some embodiments, EVs can be produced by engineered cell lines, e.g., recombinant cell lines, that express exogenous polypeptides and/or nucleic acids. In other embodiments, polypeptide-EV conjugates can be produced using EVs from other sources, for example, EVs derived from cell types including, but not limited to, platelets, reticulocytes, immune cells, intestinal epithelial cells, tumor cells, HELA cells, mesenchymal stem cells, human embryonic kidney cells (HEK cells), and all types of primary cells. In other embodiments, the EVs can be isolated from bodily fluids, e.g., milk, colostrum, etc.

In one embodiment, the EVs can comprise or consist essentially of exosomes. Exosomes can be derived from any of the foregoing cell types. For example, exosomes suitable for use in the conjugates and methods disclosed herein can be neural exosomes. Neural exosomes can be derived from neural cells, including but not limited to neural progenitor cells, neurons, astrocytes, oligodendrocytes, microglia, Schwann cells, or glioma cells. In another embodiment, the exosomes are produced synthetically.

In another embodiment, the EVs can comprise or consist essentially of ectosomes, also known as microvesicles. Microvesicles can be derived from any of the foregoing cell types. For example, microvesicles suitable for use in the conjugates and methods disclosed herein can be neural microvesicles. Neural microvesicles can be derived from neural cells, including but not limited to neural progenitor cells, neurons, astrocytes, oligodendrocytes, microglia, Schwann cells, or glioma cells. In another embodiment, the microvesicles are produced synthetically.

In some instances, the disclosed EVs can be obtained by culturing cells, such as neural cells, for a time sufficient for the cells to produce EVs. Cells used to produce EVs can be obtained, in some embodiments, from pluripotent stem cells, for example, human embryonic stem cells (hESCs) or induced pluripotent stem cells (iPSCs).

In some embodiments, EVs are isolated from neural cells, e.g., neural progenitor cells, that have been differentiated from pluripotent stem cells. Pluripotent stem cells may express one or more of the stage-specific embryonic antigens (SSEA) 3 and 4, and markers detectable using antibodies designated Tra-1-60 and Tra-1-81 (Thomson et al., Science 282:1145, 1998). Differentiation of pluripotent stem cells in vitro results in the loss of SSEA-4, Tra-1-60, and Tra-1-81 expression (if present) and increased expression of SSEA-1. Undifferentiated pluripotent stem cells typically have alkaline phosphatase activity, which can be detected by fixing the cells with 4% paraformaldehyde, and then developing with Vector Red as a substrate, as described by the manufacturer (Vector Laboratories, Burlingame Calif.) Undifferentiated pluripotent stem cells also typically express Oct-4 and TERT, as detected by antibodies or RT-PCR. In one embodiment, EVs for use in the conjugates described herein can be produced by neural progenitor cells derived from human ES cells. In another embodiment, EVs for use in the conjugates described herein can be produced by neural progenitor cells derived from human iPS cells.

The types of pluripotent stem cells that may be used include established lines of pluripotent cells derived from tissue formed after fertilization, including pre-embryonic tissue (such as, for example, a blastocyst), embryonic tissue, or fetal tissue taken any time during gestation, typically but not necessarily before approximately 10-12 weeks gestation. Non-limiting examples are established ethical lines of human embryonic stem cells or human embryonic germ cells, such as, for example the human embryonic stem cell lines WA01, WA07, and WA09 (WiCell). Also contemplated is use of the compositions of this disclosure during the initial establishment or stabilization of such cells, in which case the source cells would be primary pluripotent cells taken directly from the source tissues. Also suitable are cells taken from a pluripotent stem cell population already cultured in the absence of feeder cells. Also suitable are mutant human embryonic stem cell lines, such as, for example, BG01v (ViaCyte, San Diego, Calif.), as well as normal human embryonic stem cell lines such as WA01, WA07, WA09 (WiCell, Madison, Wis.) and BG01, BG02 (ViaCyte, San Diego, Calif.).

Human embryonic stem cells (hESCs) may be prepared by methods which are described in the in the art as described for example, by Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 1995). Alternatively, they may be obtained commercially.

iPSCs are made by dedifferentiating adult somatic cells back to a pluripotent state. iPSCs can be generated by any suitable methodology, including, but not limited to by artificial expression of four genes (c-myc, Klf4, SOX2, OCT4), or similar.

Methods for the production of human neural progenitor (hNP) cells from human embryonic stem cells (ESCs) are described, for example, in U.S. Pat. No. 7,531,354, which is hereby incorporated by reference in its entirety. Human neuroprogenitor cells (hNPs) are known to express markers associated with the earliest multipotent neural stem cells, including Nestin, Musashi-1, SOX1, SOX2 and SOX3. In one instance, the hNPs express SOX1 (SOX1+). In another instance, the hNPs express SOX2 (SOX2+). In some other instance, the hNPs express SOX3 (SOX3+). In some specific instances, the hNPs express at least one of Nestin, Musashi-1, SOX1, SOX2 and SOX3. In other instances, the hNPs express two or more of Nestin, Musashi-1, SOX1, SOX2 and SOX3. In yet another instance, the hNPs express three or more of Nestin, Musashi-1, SOX1, SOX2 and SOX3. In some instances, the hNPs express at least one of Nestin, Musashi-1, SOX1, SOX2 and SOX3 but do not express OCT4. In some other instances, the hNPs express at least two of Nestin, Musashi-1, SOX1, SOX2 and SOX3 but do not express OCT4. In yet another instance, the hNPs express at least three of Nestin, Musashi-1, SOX1, SOX2 and SOX3 but do not express OCT4. In a specific instance, the hNPs express SOX1, SOX2 and SOX3 but do not express OCT4. Neural progenitor cells may be cultured with or without feeder cells. In some instances, neuroprogenitor cells produced according to the methods presented in U.S. Pat. No. 7,531,354, are feeder cell free as well as free from embryoid bodies.

The disclosed EVs can be obtained in some instances by culturing differentiated neural cells, such as glial cells, derived directly or indirectly from pluripotent stem cells in cell culture medium under conditions and for a time sufficient to produce EVs, and isolating said EVs from the culture medium. Types of glial cells include oligodendrocytes, astrocytes, ependymal cells, Schwann cells, microglia, and satellite cells. In one instance, the differentiated neural cells (e.g., glial cells) comprise astrocytes. Differentiated neural cells that can be used include hN2™ neuronal cells (ArunA Biomedical Inc.), NeuroNet™ neurons, and AstroPro™ astrocytes (ArunA Biomedical Inc.).

EVs can be isolated from cell culture medium or tissue culture supernatant. EVs produced from cells can be collected from the culture medium by any suitable method. Typically an isolated population of EVs can be prepared from cell culture or tissue supernatant by centrifugation, size exclusion columns, microfluidic devices, polymer precipitation, filtration or combinations of these methods. For example, the EVs can be prepared as described in U.S. Patent Application Document No. 20140356382, which is hereby incorporated by reference in its entirety. For example, EVs can be prepared by differential centrifugation, that is low speed (<2,0000 g) centrifugation to pellet larger particles followed by high speed (>100,000 g) centrifugation to pellet EVs, size filtration with appropriate filters (for example, 0.22 μm filter), gradient ultracentrifugation (for example, with sucrose gradient) or a combination of these methods.

In some embodiments, the EV-producing NP cells and/or neural cells disclosed herein are cultured for about 1, 2, 3, 4, 5, 6, 7, 8, 9, 10, 11, 12, 13 or 14 days or for as long as about 1, 2, 3, 4, 5, 6, 7, 8 weeks or about 1, 2, 3, 4, 5, or 6 months, depending on the cell and its ability to produce EVs. The EV-producing cells may be cultured in suitable media and grown under conditions that are readily determined by one of ordinary skill in the art. Cell culture conditions may vary with cell type and the examples presented hereinafter illustrate suitable media and conditions. For example, CMRL 1066 medium (from Invitrogen) with exosome-depleted fetal bovine serum (e.g., at 10%) and optionally supplemented with glutamine or glutamine-containing mixtures and antibiotics could be used. Cells can be grown adhering on a surface in some embodiments, e.g. they can be grown as a monolayer to multilayers on the surface (feeder cell free) and may be grown until 30, 40, 50, 60, 70, 80, 90, 95 or 100% confluent. In other embodiments, the cells can be grown as cell aggregates or on microbeads in suspension cultures.

Cell growth media are well known in the art and comprise at least a minimum essential medium plus one or more optional components such as growth factors, ascorbic acid, glucose, non-essential amino acids, salts (including trace elements), glutamine, insulin (where indicated and not excluded), Activin A, transferrin, beta mercaptoethanol, and other agents well known in the art and as otherwise described herein. A preferred media is a low protein, serum-free based growth medium that supports neural cells. The growth factor used can be fibroblast growth factor 2 (FGF2), alone or preferably in combination with leukemia inhibitor factor (LIF). Depending on the NP or neural cells to be grown in the growth media, the inclusion of LIF is preferred but may not be required. Additional media includes basal cell media which may contain serum, for example, between about 0.1% and 20% (preferably, about 2-10%) fetal calf serum, or for defined medium, an absence of fetal calf serum and KSR, and optionally including bovine serum albumin (about 1-5%, preferably about 2%). In some instances, the medium is defined and is serum-free and has low protein content. In other instances, the media is media and supplement from ArunA which allow neural cultures to maintain a stable karyotype over multiple passages without the need for feeder cells, making them an excellent choice for a wide variety of research applications including early stage drug discovery. The components of the growth media depend on the type of neural cell to be grown, all of which are well known in the art. In one instance, a AB2™ Neural Cell Culture Media Kit is used and it contains AB2™ Basal Neural Medium and ANS™ Neural Medium Supplement. In a specific instance, the medium and supplement described in the instance above are specifically engineered for versatility to meet all neural cell culture needs. The AB2™ Basal Neural Medium and ANS™ Neural Medium Supplement can be used as the base for specialized mediums to direct differentiation of the hNP1™ line toward various neural phenotypes. Each lot of medium and supplement is pre-qualified for use by testing for cell growth, sterility, pH, osmolarity, and endotoxins.

Other agents which optionally may be added to the medium include, depending on the cell type grown in the media, for example, any one or more of nicotinamide, members of TGF-β family, including TGF-β 1, 2, and 3, Activin A, nodal, Bone Morphogen Proteins (BMP 2 to 7), serum albumin, members of the fibroblast growth factor (FGF) family, platelet-derived growth factor-AA, and —BB, platelet rich plasma, insulin growth factor (IGF-I, II, LR-IGF), growth differentiation factor (GDF-5, -6, -8, -10, 11), glucagon like peptide-I and II (GLP-I and II), GLP-1 and GLP-2 mimetobody, Exendin-4, parathyroid hormone, insulin, progesterone, aprotinin, hydrocortisone, ethanolamine, epidermal growth factor (EGF), gastrin I and II, copper chelators such as, for example, triethylene pentamine, forskolin, Na-Butyrate, betacellulin, ITS, noggin, neurite growth factor, nodal, valproic acid, trichostatin A, sodium butyrate, hepatocyte growth factor (HGF), sphingosine-1, VEGF, MG132 (EMD, CA), N2 and B27 supplements (Gibco, CA), steroid alkaloid such as, for example, cyclopamine (EMD, CA), keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, heregulin, or combinations thereof, among a number of other components. Each of these components, when included, are included in effective amounts.

In some instances, suitable media may be made from the following components, such as, for example, Dulbecco's modified Eagle's medium (DMEM), Gibco #11965-092; Knockout Dulbecco's modified Eagle's medium (KO DMEM), Gibco #10829-018; Ham's F12/50% DMEM basal medium; 200 mM L-glutamine, Gibco #15039-027; non-essential amino acid solution, Gibco 11140-050; β-mercaptoethanol, Sigma #M7522; human recombinant basic fibroblast growth factor (bFGF), Gibco #13256-029. Other suitable reagents can include Neurobasal (Gibco), BrainPhys (Stem Cell Technologies) and/or NeuroDiff (Stem Cell Technologies).

Cell media are commercially available and can be supplemented with commercially available components, including defined xeno-free components, such as those available from Invitrogen Corp. (GIBCO), Cell Applications, Inc., Biological Industries, Beth HaEmek, Israel, and Calbiochem. One of ordinary skill in the art will be able to readily modify the cell media to produce any one or more of the target cells pursuant to the present invention.

The disclosed EV-producing cells may be cultured on a layer of feeder cells that support the cells in various ways. Approaches for culturing cells on a layer of feeder cells are well known in the art. The cells may be grown on a cellular support or matrix, as adherent monolayers, or cell aggregates in suspension. In some instances, the use of a cellular support may be preferred, depending upon the cells used to produce the EVs. When used, cellular supports preferably comprise at least one substrate protein. Substrate proteins include, for example, an extracellular matrix protein, which is a protein found in the extracellular matrix, such as laminin, tenascin, thrombospondin, and mixtures thereof, which exhibit growth promoting and contain domains with homology to epidermal growth factor (EGF) and exhibit growth promoting activity. Other substrate proteins which may be used include for example, collagen, fibronectin, vibronectin, polylysine, polyornithine and mixtures thereof. In addition, gels and other materials such as methylcellulose of other gels which contain effective concentrations of one or more of these embryonic stem cell differentiation proteins may also be used. Exemplary differentiation proteins or materials which include these differentiation proteins include, for example, recombinant laminin, BD Cell-Tak™ Cell and Tissue Adhesive, BD™ FIBROGEN Human Recombinant Collagen I, BD™ FIBROGEN Human Recombinant Collagen III, BD Matrigel™ Basement Membrane Matrix, BD Matrigel™ Basement Membrane Matrix High Concentration (HC), BD™ PuraMatrix™ Peptide Hydrogel, Collagen I, Collagen I High Concentration (HC), Collagen II (Bovine), Collagen III, Collagen IV, Collagen V, and Collagen VI, among others.

Alternatively, these cells may be cultured in a culture system that is free of feeder cells, or essentially free of feeder cells, but nonetheless supports proliferation of the cells to produce EVs. The growth of cells in feeder-free culture can be supported using a medium conditioned by culturing previously with another cell type. Alternatively, the growth of EV-producing cells in feeder-free culture without differentiation can be supported using a chemically defined medium. These approaches are well known in the art. In certain embodiments of the present invention, the cells are grown in feeder cell free medium.

EVs can be harvested at various time intervals (e.g. at about 1, 2, 4, 6, 8 or 3, 6, 9, 12 day or longer intervals, depending upon the rate of production of EVs). Exemplary yields of EVs can range from at least about 1 ng EVs/1 million cells, at least about 10 ng EVs/1 million cells, at least about 50 ng EVs/1 million cells, at least about 100 ng EVs/1 million cells, at least about 500 ng EVs/1 million cells, at least about 750 ng EVs/1 million cells, at least about 800 ng EVs/1 million cells, at least about 900 ng EVs/1 million cells, at least about 1.0 μg EVs/1 million cells, at least about 1.5 μg EVs/1 million cells, at least about 2.0 μg EVs/1 million cells, at least about 2.5 μg EVs/1 million cells, at least e.g. about 3.0 μg EVs/1 million cells, at least about 5.0 μg EVs/1 million cells, and at least about 10.0 μg EVs/1 million cells, during a time period of about 24 hours to seven days of culture of proliferative and non-proliferative neural cells as otherwise described herein.

In many instances, EVs are harvested and collected by ultracentrifugation or differential centrifugation or any combination thereof, pelleted EVs are collected, and, optionally, collected pelleted EVs are washed with a suitable medium. For example, a preparation of EVs can be prepared from cell culture or tissue supernatant by centrifugation, filtration or combinations of these methods. In some embodiments, the EVs can be prepared by differential centrifugation, that is low speed (<2,0000 g) centrifugation to pellet larger particles followed by high speed (>100,000 g) centrifugation to pellet EVs, size filtration with appropriate filters (for example, 0.22 μm filter), gradient ultracentrifugation (for example, with sucrose gradient) or a combination of these methods. EVs may be purified by differential centrifugation, micro and ultra-filtration, polymeric precipitation, microfluidic separation, immunocapture and size-exclusion chromatography. These and/or related methods for isolating and purifying EVs are described by Théry, et al., Current Protocols in Cell Biology, (2006) 3.221-3.22.29, copyright 2006 by John Wiley & Sons, Inc.; Sokolova, et al., Colloids and Surfaces B: Biointerfaces, 2011, 87, 146-150; Wiklander, et al., Journal of Extracellular Vesicles, 2015, 4, 26316, pp. 1-13; and Boing, et al., Journal of Extracellular Vesicles, 2014, 3, 23430, pp. 1-11. Other methods for isolation may be developed such as electrical field radiofrequency and acoustics.

Methods of manufacturing synthetic vesicles, such as synthetic exosomes, are known in the art. Such methods may be used to produce synthetic vesicles suitable for use in the compositions and methods provided herein. It is noted that the contents of EVs, i.e., EVs in which the lipid bilayer has been removed or eliminated and the contents obtained, may also be used to engineer artificial EVs.

(ii) Polypeptide-EV Conjugates

In one aspect, the invention provides a composition comprising a polypeptide-EV conjugate comprising an extracellular vesicle derived from a neural cell (e.g., neural progenitor cell or neural stem cell), wherein the polypeptide is conjugated to the vesicle surface using click chemistry. In some embodiments, the polypeptide is an antibody, or antigen binding portion thereof (referred to herein as an “antibody-EV (Ab-EV) conjugate”). The polypeptide-EV conjugate (e.g., Ab-EV conjugate) can comprise an extracellular vesicle derived from neural cells (e.g., neural progenitor cells or neural stem cells) that is conjugated on the vesicle surface to a polypeptide (e.g., antibody, or antigen-binding portion thereof). The polypeptide (e.g., antibody) can be conjugated to the vesicle surface using click chemistry. In such embodiments, the polypeptide (e.g., antibody) can be coupled to the vesicle by way of a linker generated from a reaction between two complementary click chemistry functional groups. Additional features of polypeptide-EV conjugates are provided below.

To prepare the polypeptide-EV (e.g., Ab-EV) conjugates provided herein, an antibody or antigen binding portion thereof can be conjugated to the EV using a variety of conjugation methods.

In some embodiments, the polypeptide coupled to an EV is an antibody, or antigen binding portion thereof. The antibody may be, for example, a monoclonal antibody, polyclonal antibody, a multispecific antibody (e.g., a bispecific antibody), nanobody, monobody, and antibody fragment that exhibits the desired antigen-binding activity. The antibody, or antigen-binding portion thereof, can be in the form of a full-length (intact) antibody, bispecific antibody, dual variable domain antibody, multiple chain or single chain antibody, and/or antigen binding fragments thereof, including but not limited to Fab, Fab′, (Fab′)2, Fv, scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the antibody or antigen binding portion thereof is a humanized antibody or antigen binding portion thereof. In other embodiments, the antibody or antigen-binding portion thereof is a fully human antibody or antigen binding portion thereof.

In some embodiments, an antibody mimetic is conjugated to the EVs provided herein. Examples of antibody mimetics include, but are not limited to, an adnectin (i.e., fibronectin based binding molecules), an affilin, an affimer, an affitin, an alphabody, an affibody, DARPins, an anticalin, an avimer, a fynomer, a Kunitz domain peptide, a monobody, a nanoCLAMP, a nanobody, a unibody, a versabody, an aptamer, and a peptidic molecule all of which employ binding structures that, while they mimic traditional antibody binding, are generated from and function via distinct mechanisms.

The polypeptide (e.g., antibody or antigen binding portion thereof) can be conjugated to the surface of the EV using “click chemistry” (see, e.g., Kolb, H. C.; Finn, M. G.; Sharpless, K. B. Angewandte Chemie, International Edition 2001, 40, 2004-2021; Kolb, H. C.; Sharpless, K. B. Drug Discovery Today 2003, 8, 1128-1137; the disclosures of which are incorporated herein by reference in their entirety). Any suitable click reaction can be used to link the polypeptide to the EV surface. Click chemistry reactions are advantageous as they are typically fast, modular, efficient, often do not produce toxic waste products, can be done with water as a solvent, and can be set up to be stereospecific.

The term “click functional group”, recited herein is used interchangeably with the terms “click chemistry reagent” or “click reagent” to refer to a reagent that can rapidly and selectively react (“click”, e.g., via a cycloaddition reaction) with its counterpart click reagent under mild conditions in aqueous solution. Mild conditions can include neutral pH, aqueous solution and ambient temperature, with low reactant concentrations. Examples of click functional groups include azide, alkene, alkyne, dibenzocyclooctyne (DBCO), transcyclooctene, nitrone, nitrilimine, nitrile oxide, isonitrile, tetrazole and tetrazine groups. Exemplary click reactions include but are not limited to Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy. In some embodiments, the linker that couples the antibody or antigen binding portion thereof to the EV is generated from a reaction between two complementary click functional groups (“click linker”).

In one embodiment, a polypeptide (e.g., antibody or antigen binding portion thereof) or the EV of the polypeptide-EV conjugates herein can comprise a click functional group which is a click functional unsaturated group. Examples are dipolarophiles, such as alkenes and alkynes, as well as molecules which comprise related heteroatom functional groups, for example carbonyls and nitriles. Other click functional groups which can be used as reactants for click functional unsaturated groups are dienes such as tetrazine and tetrazole.

In another embodiment, a polypeptide (e.g., antibody or antigen binding portion thereof) or the EV of the polypeptide-EV conjugates herein can comprise a click functional dipolar group. Click functional dipolar groups are understood as being those compounds which comprise one or more heteroatoms and which have at least one mesomeric structure which represents a charged dipole. Examples of click functional dipolar groups are linear 1,3-dipolar groups, for example azide, nitrile oxide, diazoalkane, nitrilimine and nitrone.

In certain embodiments, an EV-Ab conjugate is prepared by reacting an antibody or antigen binding portion thereof comprising a first click functional group with an EV comprising a second click functional group that are known to undergo a click chemistry reaction. Non-limiting examples of complementary pairs of functional groups that may be used to enable conjugation of the antibody or antigen binding portion thereof and EV are shown in Table 1.

TABLE 1
Examples of complementary click functional group
pairs for formation of Ab-EV conjugates

| | EV Functional | Antibody Functional |
|---|---|---|
| Conjugation Reaction | Group | Group |
| Copper(I)-catalyzed azide- | Azide | Alkyne |
| alkyne cycloaddition | Alkyne | Azide |
| Strain-promoted azide- | Azide (strain- | Alkyne |
| alkyne cycloaddition | promoted) | |
| | Alkyne | Azide (strain- |
| | | promoted) |
| Strain-promoted alkyne- | Alkyne (strain- | Nitrone |
| nitrone cycloaddition | promoted) | |
| | Nitrone | Alkyne (strain- |
| | | promoted) |
| Alkene and azide [3 + 2] | Alkene | Azide |
| cycloaddition | Azide | Alkene |
| Alkene and tetrazine | Alkene | Tetrazine |
| inverse-demand Diels- | Tetrazine | Alkene |
| Alder | | |
| Alkene and tetrazole | Alkene | Tetrazole |
| photoclick | Tetrazole | Alkene |

Other exemplary pairs of click reagents that are well known to persons of skill in the art include, but are not limited to, azide and dibenzocyclooctyne (DBCO, also known as DIBO), tetrazine and transcyclooctene, and tetrazine and norbornene.

In one exemplary embodiment, EVs labeled with azide can be coupled to an antibody or antigen binding portion thereof labeled with DBCO. In another embodiment, EVs labeled with DBCO can be coupled to an antibody or antigen binding portion thereof labeled with azide. In such embodiments, the antibody or antigen binding portion thereof is conjugated to the EVs via a click linker formed from reaction of azide and DBCO.

In another exemplary embodiment, EVs labeled with tetrazine can be coupled to an antibody or antigen binding portion thereof labeled with transcyclooctene. In another embodiment, EVs labeled with transcyclooctene can be coupled to an antibody or antigen binding portion thereof labeled with tetrazine. In such embodiments, the antibody or antigen binding portion thereof is conjugated to the EVs via a click linker formed from reaction of tetrazine and transcyclooctene.

In another exemplary embodiment, EVs labeled with tetrazine can be coupled to an antibody or antigen binding portion thereof labeled with norbornene. In another embodiment, EVs labeled with norbornene can be coupled to an antibody or antigen binding portion thereof labeled with tetrazine. In such embodiments, the antibody or antigen binding portion thereof is conjugated to the EVs via a click linker formed from reaction of tetrazine and norbornene.

Exemplary reactions that may be used to conjugate the antibody to an EV provided herein include the copper catalyzed reaction of an azide and alkyne to form a triazole (Huisgen 1, 3-dipolar cycloaddition), reaction of a diene and dienophile (Diels-Alder), strain-promoted azide-alkyne cycloaddition, strain-promoted alkyne-nitrone cycloaddition, reaction of a strained alkene with an azide, tetrazine or tetrazole, alkene and azide [3+2] cycloaddition, alkene and tetrazine inverse-demand Diels-Alder, or an alkene and tetrazole photoreaction. In some embodiments the reaction may be performed in an aqueous environment. In certain embodiments, the reaction is a copper-catalyzed or a ruthenium-catalyzed reaction (e.g., to initiate a reaction between azides and alkynes, e.g., in a azide-alkyne cycloaddition reaction). Alternatively, the reaction may be a copper-free reaction (e.g., a strain-promoted azide-alkyne cycloaddition reaction, strain-promoted alkyne-nitrone cycloaddition).

Methods of incorporating click functional groups onto proteins and EVs are described in, for example, Smyth et al. “Surface functionalization of exosomes using click chemistry.” Bioconjugate chemistry 25.10 (2014): 1777-1784; Wang et al. “Integrating protein engineering and bioorthogonal click conjugation for extracellular vesicle modulation and intracellular delivery.” PLoS One 10.11 (2015): e0141860; and Lee et al. “Facile metabolic glycan labeling strategy for exosome tracking.” Biochimica et Biophysica Acta (BBA)-General Subjects 1862.5 (2018): 1091-1100, each of which is hereby incorporated by reference in its entirety.

The number of proteins, such as antibodies, conjugated to the EVs can be varied by altering the degree of click conjugation of the EVs. The term “degree of click conjugation”, which may be used interchangeably with the term “degree of substitution” or “DS” refers to the average number of click reagents per EV. The degree of click substitution may be varied by varying the number of molar equivalents of click reagent to the concentration of EVs in the click conjugation reaction. For example, the click conjugation reaction may comprise about 1 to about 5000 molar equivalents of a click reagent, e.g., about 1, about 5, about 10, about 20, about 50, about 100, about 150, about 200, about 250, about 300, about 350, about 400, about 450, about 500, about 550, about 600, about 650, about 700, about 750, about 800, about 850, about 900, about 950, about 1000, about 1200, about 1500, about 1800, about 2000, about 2200, about 2500, about 2800, about 3000, about 3200, about 3500, about 4000, about 4200, about 4500 or about 5000 molar equivalents of the click reagent. Ranges intermediate to the recited values are also intended to be part of this invention. For example, the click conjugation reaction may comprise about 1 to about 50, about 10 to about 100, about 150 to about 250, about 200 to about 500, about 400 to about 800, about 700 to about 1000, about 1200 to about 1600, about 1500 to about 2000, about 1800 to about 3500, or about 3200 to about 5000 molar equivalents of a click reagent.

In some embodiments, the EV conjugated to a click reagent comprises a degree of click substitution that is about 0.01% to about 100%, e.g., about 0.01% to about 0.5%, about 0.1% to about 5%, about 1% to about 10%, about 5% to about 15%, about 10% to about 20%, about 15% to about 25%, about 20% to about 35%, about 30% to about 40%, about 35% to about 50%, about 40% to about 60%, about 50% to about 75%, about 70% to about 90% or about 85% to about 100%. For example, the EV of the invention conjugated to a click reagent may be about 0.01%, about 0.05%, about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

Any antibody or antigen binding portion thereof can be conjugated to EVs derived from neural cells using the methods provided herein. In some embodiments, the antibody is a chimeric antibody, or antigen binding portion thereof. In other embodiments, the antibody is a humanized antibody, or antigen binding portion thereof. In other embodiments, the antibody is a fully human antibody, or antigen binding portion thereof. In exemplary embodiments, the antibody or antigen binding fragment thereof specifically binds to a target protein that is expressed in the brain or central nervous system. For example, in some embodiments, the antibody or antigen binding portion thereof can specifically bind to an amyloid beta polypeptide (Aβ), e.g., a human amyloid beta polypeptide, associated with Alzheimer's Disease.

In an exemplary embodiment, the antibody-EV conjugates provided herein can comprise the anti-amyloid beta antibody solanezumab, or an antigen binding portion thereof. Solanezumab is a humanized IgG1 monoclonal antibody targeting the mid-domain of Aβ. Solanezumab (also known as 10D5 and m266) is described in, e.g., U.S. Pat. Nos. 7,320,790, 7,195,761, 8,105,597, 8,591,894, and 8,623,365, each of which is incorporated by reference in its entirety.

In one embodiment, the antibody-EV conjugates described herein include an anti-amyloid beta antibody or antigen binding portion thereof that comprises the variable heavy and/or light chain regions of solanezumab. In one embodiment, the anti-amyloid beta antibody comprises a heavy chain variable region comprising a VH CDR1, VH CDR2 and VH CDR3 of solanezumab, and/or a light chain variable region comprising a VL CDR1, VL CDR2 and VL CDR3 of solanezumab.

The heavy chain and light chain amino acid sequences of solanezumab are described in Table 2. The CDR regions (CDR1, CDR2, and CDR3) of solanezumab are highlighted in bold. The heavy chain variable region and light chain variable region of solanezumab are underlined.

TABLE 2
Amino Acid Sequence of Solanezumab

| SEQ | | |
|---|---|---|
| ID | SEQUENCE | |
| NO: | DESCRIPTION | AMINO ACID SEQUENCE |
| 1 | Solanezumab | EVQLVESGGGLVQPGGSLRLSCAASGFTFSRYSMSWV |
| | heavy chain | RQAPGKGLELVAQINSVGNSTYYPDTVKGRFTISRDNA |
| | | KNTLYLQMNSLRAEDTAVYYCASGDYWGQGTLVTVS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSCDKTHTCPPCPAP |
| | | ELLGGPSVFLFPPKPKDTLMISRTPEVTCVVVDVSHEDP |
| | | EVKFNWYVDGVEVHNAKTKPREEQYNSTYRVVSVLTV |
| | | LHQDWLNGKEYKCKVSNKALPAPIEKTISKAKGQPREP |
| | | QVYTLPPSRDELTKNQVSLTCLVKGFYPSDIAVEWESN |
| | | GQPENNYKTTPPVLDSDGSFFLYSKLTVDKSRWQQGN |
| | | VFSCSVMHEALHNHYTQKSLSLSPG |
| 2 | Solanezumab | EVQLVESGGGLVQPGGSLRLSCAASGFTFSRYSMSWV |
| | heavy chain | RQAPGKGLELVAQINSVGNSTYYPDTVKGRFTISRDNA |
| | variable region | KNTLYLQMNSLRAEDTAVYYCASGDYWGQGTLVTVS |
| | | SA |
| 3 | Solanezumab | GFTFSRY |
| | VH CDR1 | |
| 4 | Solanezumab | NSVG |
| | VH CDR2 | |
| 5 | Solanezumab | GDY |
| | VH CDR3 | |
| 6 | Solanezumab | DVVMTQSPLSLPVTLGQPASISCRSSQSLIYSDGNAYLH |
| | light chain | WFLQKPGQSPRLLIYKVSNRFSGVPDRFSGSGSGTDFTL |
| | | KISRVEAEDVGVYYCSQSTHVPWTFGQGTKVEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 7 | Solanezumab | DVVMTQSPLSLPVTLGQPASISCRSSQSLIYSDGNAYLH |
| | light chain | WFLQKPGQSPRLLIYKVSNRFSGVPDRFSGSGSGTDFTL |
| | variable region | KISRVEAEDVGVYYCSQSTHVPWTFGQGTKVEIKR |
| 8 | Solanezumab | QSLIYSDGNAYLH |
| | VL CDR1 | |
| 9 | Solanezumab | KVSNRF |
| | VL CDR2 | |
| 10 | Solanezumab | QSTHVPWT |
| | VL CDR3 | |

In one embodiment, the antibody-EV conjugate comprises an antibody or antigen binding portion thereof comprising one, two, or three heavy chain CDR regions set forth in Table 2, or a sequence having at least 95%, 96%, 97%, 98%, or 99% identity thereto, and/or one, two, or three light chain CDR regions set forth in Table 2, or a sequence having at least 95%, 96%, 97%, 98%, or 99% identity thereto. In one embodiment, the antibody or antigen binding portion thereof comprises a VH CDR1 of SEQ ID NO:3, a VH CDR2 of SEQ ID NO:4, and a VH CDR3 of SEQ ID NO:5, and/or a VL CDR1 of SEQ ID NO:8, a VL CDR2 of SEQ ID NO:9, and a VL CDR3 of SEQ ID NO:10.

In one embodiment, the antibody-EV conjugate has a heavy chain variable region (HCVR) comprising at least 95%, at least 96%, at least 97%, at least 98%, at least 99% or 100% identity to the amino acid sequence set forth in SEQ ID NO:2, and/or a light chain comprising at least 95%, at least 96%, at least 97%, at least 98%, at least 99% or 100% identity to the amino acid sequence set forth in SEQ ID NO:7.

In one embodiment, the antibody-EV conjugate has a heavy chain comprising at least 95%, at least 96%, at least 97%, at least 98%, at least 99% or 100% identity to the amino acid sequence set forth in SEQ ID NO:1, and/or a light chain comprising at least 95%, at least 96%, at least 97%, at least 98%, at least 99% or 100% identity to the amino acid sequence set forth in SEQ ID NO:6.

In another exemplary embodiment, the antibody-EV conjugates provided herein can comprise the anti-amyloid beta antibody aducanumab, or an antigen binding portion thereof. Aducanumab is a fully human IgG1 monoclonal antibody that recognizes a conformational epitope on aggregated forms of Aβ. Aducanumab (also known as BIIB037 and BART) is described in, e.g., US20150315267, US20180333487, WO2017211827, and WO2019040612, each of which is incorporated by reference in its entirety.

In one embodiment, the antibody-EV conjugates described herein include an anti-amyloid beta antibody or antigen binding portion thereof that comprises the variable heavy and/or light chain regions of aducanumab. In one embodiment, the anti-amyloid beta antibody comprises a heavy chain variable region comprising a VH CDR1, VH CDR2 and VH CDR3 of aducanumab, and/or a light chain variable region comprising a VL CDR1, VL CDR2 and VL CDR3 of aducanumab.

The heavy chain and light chain amino acid sequences of aducanumab are described in Table 3. The CDR regions (CDR1, CDR2, and CDR3) of aducanumab are highlighted in bold. The heavy chain variable region and light chain variable region of aducanumab are underlined.

TABLE 3
Amino Acid Sequence of Aducanumab

| SEQ | | |
|---|---|---|
| ID | SEQUENCE | |
| NO: | DESCRIPTION | AMINO ACID SEQUENCE |
| 11 | Aducanumab | QVQLVESGGGVVQPGRSLRLSCAASGFAFSSYGMHWVR |
| | heavy chain | QAPGKGLEWVAVIWFDGTKKYYTDSVKGRFTISRDNSK |
| | | NTLYLQMNTLRAEDTAVYYCARDRGIGARRGPYYMDV |
| | | WGKGTTVTVSSASTKGPSVFPLAPSSKSTSGGTAALGCL |
| | | VKDYFPEPVTVSWNSGALTSGVHTFPAVLQSSGLYSLSS |
| | | VVTVPSSSLGTQTYICNVNHKPSNTKVDKRVEPKSCDKT |
| | | HTCPPCPAPELLGGPSVFLFPPKPKDTLMISRTPEVTCVVV |
| | | DVSHEDPEVKFNWYVDGVEVHNAKTKPREEQYNSTYRV |
| | | VSVLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQQ |
| | | GNVFSCSVMHEALHNHYTQKSLSLSPG |
| 12 | Aducanumab | QVQLVESGGGVVQPGRSLRLSCAASGFAFSSYGMHWVR |
| | heavy chain | QAPGKGLEWVAVIWFDGTKKYYTDSVKGRFTISRDNSK |
| | variable | NTLYLQMNTLRAEDTAVYYCARDRGIGARRGPYYMDV |
| | region | WGKGTTVTVSSA |
| 13 | Aducanumab | GFAFSSY |
| | VH CDR1 | |
| 14 | Aducanumab | WFDGTK |
| | VH CDR2 | |
| 15 | Aducanumab | DRGIGARRGPYYMDV |
| | VH CDR3 | |
| 16 | Aducanumab | DIQMTQSPSSLSASVGDRVTITCRASQSISSYLNWYQQKP |
| | light chain | GKAPKLLIYAASSLQSGVPSRFSGSGSGTDFTLTISSLQPE |
| | | DFATYYCQQSYSTPLTFGGGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| 17 | Aducanumab | DIQMTQSPSSLSASVGDRVTITCRASQSISSYLNWYQQKP |
| | light chain | GKAPKLLIYAASSLQSGVPSRFSGSGSGTDFTLTISSLQPE |
| | variable region | DFATYYCQQSYSTPLTFGGGTKVEIKR |
| 18 | Aducanumab | QSISSYLN |
| | VL CDR1 | |
| 19 | Aducanumab | AASSLQS |
| | VL CDR2 | |
| 20 | Aducanumab | QQSYSTPLT |
| | VL CDR3 | |

In one embodiment, the antibody-EV conjugate comprises an antibody or antigen binding portion thereof comprising one, two, or three heavy chain CDR regions set forth in Table 3, or a sequence having at least 95%, 96%, 97%, 98%, or 99% identity thereto, and/or one, two, or three light chain CDR regions set forth in Table 3, or a sequence having at least 95%, 96%, 97%, 98%, or 99% identity thereto. In one embodiment, the antibody or antigen binding portion thereof comprises a VH CDR1 of SEQ ID NO:13, a VH CDR2 of SEQ ID NO:14, and a VH CDR3 of SEQ ID NO:15, and/or a VL CDR1 of SEQ ID NO:18, a VL CDR2 of SEQ ID NO:19, and a VL CDR3 of SEQ ID NO:20.

In one embodiment, the antibody-EV conjugate has a heavy chain variable region (HCVR) comprising at least 95%, at least 96%, at least 97%, at least 98%, at least 99% or 100% identity to the amino acid sequence set forth in SEQ ID NO:12, and/or a light chain variable region (LCVR) comprising at least 95%, at least 96%, at least 97%, at least 98%, at least 99% or 100% identity to the amino acid sequence set forth in SEQ ID NO:17.

In one embodiment, the antibody-EV conjugate has a heavy chain comprising at least 95%, at least 96%, at least 97%, at least 98%, at least 99% or 100% identity to the amino acid sequence set forth in SEQ ID NO:11, and/or a light chain comprising at least 95%, at least 96%, at least 97%, at least 98%, at least 99% or 100% identity to the amino acid sequence set forth in SEQ ID NO:16.

In another embodiment, the antibody, or antigen-binding fragment thereof, comprises a heavy chain variable region that comprises an amino acid sequence having at least 95% identity to an anti-amyloid beta antibody provided herein, e.g., at least 95%, 96%, 97%, 98%, 99%, or 100% identity to an anti-amyloid beta antibody provided herein. In certain embodiments, an antibody comprises a modified heavy chain (HC) variable region comprising an HC variable domain of an anti-amyloid beta antibody herein, or a variant thereof, which variant (i) differs from the anti-amyloid beta antibody in 1, 2, 3, 4 or 5 amino acids substitutions, additions or deletions; (ii) differs from the anti-amyloid beta antibody in at most 5, 4, 3, 2, or 1 amino acids substitutions, additions or deletions; (iii) differs from the anti-amyloid beta antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-amyloid beta antibody, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of the anti-amyloid beta antibody, while retaining the amyloid beta binding specificity of the antibody.

In another exemplary embodiment, the antibody-EV conjugates provided herein can comprise an antibody or antigen binding portion thereof that specifically binds to a human Programmed Cell Death 1 (PD1) protein. Antibody-EV conjugates comprising an antibody or antigen binding portion thereof that specifically binds to PD1 can be used in the treatment of diseases or disorders in which PD1 is implicated, for example, in the treatment of cancer, including but not limited to glioblastoma multiforme. In an exemplary embodiment, the antibody-EV conjugates provided herein can comprise the anti-PD1 antibody nivolumab, or an antigen binding portion thereof. Nivolumab is a human IgG4 monoclonal antibody targeting PD1. Nivolumab is described in, e.g., U.S. Pat. Nos. 8,008,449, 8,168,179, and 9,387,247, each of which is incorporated by reference in its entirety.

In one embodiment, the antibody-EV conjugates described herein include an anti-PD1 antibody or antigen binding portion thereof that comprises the variable heavy and/or light chain regions of nivolumab. In one embodiment, the PD1 antibody comprises a heavy chain variable region comprising a VH CDR1, VH CDR2 and VH CDR3 of nivolumab, and/or a light chain variable region comprising a VL CDR1, VL CDR2 and VL CDR3 of nivolumab. The heavy chain and light chain amino acid sequences of nivolumab are described in Table 4.

TABLE 4
Amino Acid Sequence of Nivolumab

| SEQ | | |
|---|---|---|
| ID | SEQUENCE | |
| NO: | DESCRIPTION | AMINO ACID SEQUENCE |
| 21 | Nivolumab | QVQLVESGGGVVQPGRSLRLDCKASGITFSNSGMHWVR |
| | heavy chain | QAPGKGLEWVAVIWYDGSKRYYADSVKGRFTISRDNSK |
| | | NTLFLQMNSLRAEDTAVYYCATNDDYWGQGTLVTVSS |
| | | ASTKGPSVFPLAPCSRSTSESTAALGCLVKDYFPEPVTVS |
| | | WNSGALTSGVHTFPAVLQSSGLYSLSSVVTVPSSSLGTK |
| | | TYTCNVDHKPSNTKVDKRVESKYGPPCPPCPAPEFLGGP |
| | | SVFLFPPKPKDTLMISRTPEVTCVVVDVSQEDPEVQFNW |
| | | YVDGVEVHNAKTKPREEQFNSTYRVVSVLTVLHQDWL |
| | | NGKEYKCKVSNKGLPSSIEKTISKAKGQPREPQVYTLPPS |
| | | QEEMTKNQVSLTCLVKGFYPSDIAVEWESNGQPENNYK |
| | | TTPPVLDSDGSFFLYSRLTVDKSRWQEGNVFSCSVMHE |
| | | ALHNHYTQKSLSLSLGK |
| 22 | Nivolumab | QVQLVESGGGVVQPGRSLRLDCKASGITFSNSGMHWVR |
| | heavy chain | QAPGKGLEWVAVIWYDGSKRYYADSVKGRFTISRDNSK |
| | variable region | NTLFLQMNSLRAEDTAVYYCATNDDYWGQGTLVTVSS |
| | | A |
| 23 | Nivolumab | GITFSNS |
| | VH CDR1 | |
| 24 | Nivolumab | WYDGSK |
| | VH CDR2 | |
| 25 | Nivolumab | NDDY |
| | VH CDR3 | |
| 26 | Nivolumab | EIVLTQSPATLSLSPGERATLSCRASQSVSSYLAWYQQKP |
| | light chain | GQAPRLLIYDASNRATGIPARFSGSGSGTDFTLTISSLEPE |
| | | DFAVYYCQQSSNWPRTFGQGTKVEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| 27 | Nivolumab | EIVLTQSPATLSLSPGERATLSCRASQSVSSYLAWYQQKP |
| | light chain | GQAPRLLIYDASNRATGIPARFSGSGSGTDFTLTISSLEPE |
| | variable region | DFAVYYCQQSSNWPRTFGQGTKVEIKR |
| 28 | Nivolumab | QSVSSYLA |
| | VL CDR1 | |
| 29 | Nivolumab | DASNRAT |
| | VL CDR2 | |
| 30 | Nivolumab | QQSSNWPRT |
| | VL CDR3 | |

In one embodiment, the antibody-EV conjugate comprises an antibody or antigen binding portion thereof comprising one, two, or three heavy chain CDR regions set forth in Table 4, or a sequence having at least 95%, 96%, 97%, 98%, or 99% identity thereto, and/or one, two, or three light chain CDR regions set forth in Table 4, or a sequence having at least 95%, 96%, 97%, 98%, or 99% identity thereto. In one embodiment, the antibody or antigen binding portion thereof comprises a VH CDR1 of SEQ ID NO:23, a VH CDR2 of SEQ ID NO:24, and a VH CDR3 of SEQ ID NO:25, and/or a VL CDR1 of SEQ ID NO:28, a VL CDR2 of SEQ ID NO:29, and a VL CDR3 of SEQ ID NO:30.

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:22, and/or a light chain variable region (LCVR) comprising at least 95%, at least 96%, at least 97%, at least 98%, at least 99% or 100% identity to the amino acid sequence set forth in SEQ ID NO:27.

In one embodiment, the antibody-EV conjugate comprises a heavy chain comprising at least 95%, at least 96%, at least 97%, at least 98%, at least 99% or 100% identity to the amino acid sequence set forth in SEQ ID NO:21, and/or a light chain comprising at least 95%, at least 96%, at least 97%, at least 98%, at least 99% or 100% identity to the amino acid sequence set forth in SEQ ID NO:26.

In another exemplary embodiment, the antibody-EV conjugates provided herein can comprise an antibody or antigen binding portion thereof that specifically binds to a human vascular endothelial growth factor (VEGF) protein, associated with cancer. Antibody-EV conjugates comprising an antibody or antigen binding portion thereof that specifically binds to VEGF can be used in the treatment of diseases or disorders in which VEGF is implicated, for example, in the treatment of cancer, including but not limited to glioblastoma multiforme. In an exemplary embodiment, the antibody-EV conjugates provided herein can comprise the anti-VEGF antibody bevacizumab, or an antigen binding portion thereof. Bevacizumab is a recombinant humanized monoclonal antibody targeting VEGF. Bevacizumab is described in, e.g., U.S. Pat. Nos. 6,632,926, 7,169,901, and 7,575,893, each of which is incorporated by reference in its entirety.

In one embodiment, the antibody-EV conjugates described herein include an anti-VEGF antibody or antigen binding portion thereof that comprises the variable heavy and/or light chain regions of bevacizumab. In one embodiment, the VEGF antibody comprises a heavy chain variable region comprising a VH CDR1, VH CDR2 and VH CDR3 of bevacizumab, and/or a light chain variable region comprising a VL CDR1, VL CDR2 and VL CDR3 of bevacizumab. The heavy chain and light chain amino acid sequences of bevacizumab are described in Table 5.

TABLE 5
Amino Acid Sequence of Bevacizumab

| SEQ | | |
|---|---|---|
| ID | SEQUENCE | |
| NO: | DESCRIPTION | AMINO ACID SEQUENCE |
| 31 | Bevacizumab | EVQLVESGGGLVQPGGSLRLSCAASGYTFTNYGMNWV |
| | heavy chain | RQAPGKGLEWVGWINTYTGEPTYAADFKRRFTFSLDTS |
| | | KSTAYLQMNSLRAEDTAVYYCAKYPHYYGSSHWYFDV |
| | | WGQGTLVTVSSASTKGPSVFPLAPSSKSTSGGTAALGCL |
| | | VKDYFPEPVTVSWNSGALTSGVHTFPAVLQSSGLYSLSS |
| | | VVTVPSSSLGTQTYICNVNHKPSNTKVDKKVEPKSCDKT |
| | | HT |
| 32 | Bevacizumab | EVQLVESGGGLVQPGGSLRLSCAASGYTFTNYGMNWV |
| | heavy chain | RQAPGKGLEWVGWINTYTGEPTYAADFKRRFTFSLDTS |
| | variable region | KSTAYLQMNSLRAEDTAVYYCAKYPHYYGSSHWYFDV |
| | | WGQGTLVTVSSA |
| 33 | Bevacizumab | GYTFTNY |
| | VH CDR1 | |
| 34 | Bevacizumab | NTYTGE |
| | VH CDR2 | |
| 35 | Bevacizumab | YPHYYGSSHWYFDV |
| | VH CDR3 | |
| 36 | Bevacizumab | DIQMTQSPSSLSASVGDRVTITCSASQDISNYLNWYQQK |
| | light chain | PGKAPKVLIYF |
| | | TSSLHSGVPSRFSGSGSGTDFTLTISSLQPEDFATYYCQQ |
| | | YSTVPWTFGQ |
| | | GTKVEIKRTVAAPSVFIFPPSDEQLKSGTASVVCLLNNFY |
| | | PREAKVQWKV |
| | | DNALQSGNSQESVTEQDSKDSTYSLSSTLTLSKADYEKH |
| | | KVYACEVTHQG |
| | | LSSPVTKSFNRGEC |
| 37 | Bevacizumab | DIQMTQSPSSLSASVGDRVTITCSASQDISNYLNWYQQK |
| | light chain | PGKAPKVLIYF |
| | variable region | TSSLHSGVPSRFSGSGSGTDFTLTISSLQPEDFATYYCQQ |
| | | YSTVPWTFGQ |
| | | GTKVEIKR |
| 38 | Bevacizumab | QDISNYLN |
| | VL CDR1 | |
| 39 | Bevacizumab | FTSSLHS |
| | VL CDR2 | |
| 40 | Bevacizumab | QQYSTVPWT |
| | VL CDR3 | |

In one embodiment, the antibody-EV conjugate comprises an antibody or antigen binding portion thereof comprising one, two, or three heavy chain CDR regions set forth in Table 5, or a sequence having at least 95%, 96%, 97%, 98%, or 99% identity thereto, and/or one, two, or three light chain CDR regions set forth in Table 5, or a sequence having at least 95%, 96%, 97%, 98%, or 99% identity thereto. In one embodiment, the antibody or antigen binding portion thereof comprises a VH CDR1 of SEQ ID NO:33, a VH CDR2 of SEQ ID NO:34, and a VH CDR3 of SEQ ID NO:35, and/or a VL CDR1 of SEQ ID NO:38, a VL CDR2 of SEQ ID NO:39, and a VL CDR3 of SEQ ID NO:40.

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:32, and/or a light chain variable region (LCVR) comprising at least 95%, at least 96%, at least 97%, at least 98%, at least 99% or 100% identity to the amino acid sequence set forth in SEQ ID NO:37.

In one embodiment, the antibody-EV conjugate comprises a heavy chain comprising at least 95%, at least 96%, at least 97%, at least 98%, at least 99% or 100% identity to the amino acid sequence set forth in SEQ ID NO:31, and/or a light chain comprising at least 95%, at least 96%, at least 97%, at least 98%, at least 99% or 100% identity to the amino acid sequence set forth in SEQ ID NO:36.

In another exemplary embodiment, the antibody-EV conjugates provided herein can comprise an antibody or antigen binding portion thereof that specifically binds to a human B-lymphocyte antigen CD20 (CD20) protein. Antibody-EV conjugates comprising an antibody or antigen binding portion thereof that specifically binds to CD20 can be used in the treatment of diseases or disorders in which CD20 is implicated, for example, in the treatment of multiple sclerosis (MS), including relapsing MS, primary progressive MS, and secondary progressive MS. In an exemplary embodiment, the antibody-EV conjugates provided herein can comprise the anti-CD20 antibody ocrelizumab, or an antigen binding portion thereof. Ocrelizumab is a humanized IgG1 monoclonal antibody targeting CD20. Ocrelizumab is described in, e.g., U.S. Pat. Nos. 5,500,362, 5,677,180, and U.S. Pat. No. 7,799,900, each of which is incorporated by reference in its entirety.

In one embodiment, the antibody-EV conjugates described herein include an anti-CD20 antibody or antigen binding portion thereof that comprises the variable heavy and/or light chain regions of ocrelizumab. In one embodiment, the CD20 antibody comprises a heavy chain variable region comprising a VH CDR1, VH CDR2 and VH CDR3 of ocrelizumab, and/or a light chain variable region comprising a VL CDR1, VL CDR2 and VL CDR3 of ocrelizumab. The heavy chain and light chain amino acid sequences of ocrelizumab are described in Table 6.

TABLE 6
Amino Acid Sequence of Ocrelizumab

| SEQ | | |
|---|---|---|
| ID | SEQUENCE | |
| NO: | DESCRIPTION | AMINO ACID SEQUENCE |
| 41 | Ocrelizumab | EVQLVESGGGLVQPGGSLRLSCAASGYTFTSYNMHWVR |
| | heavy chain | QAPGKGLEWVGAIYPGNGDTSYNQKFKGRFTISVDKSK |
| | | NTLYLQMNSLRAEDTAVYYCARVVYYSNSYWYFDVW |
| | | GQGTLVTVSSASTKGPSVFPLAPSSKSTSGGTAALGCLV |
| | | KDYFPEPVTVSWNSGALTSGVHTFPAVLQSSGLYSLSSW |
| | | TVPSSSLGTQTYICNVNHKPSNTKVDKKVEPKSCDKTHT |
| | | CPPCPAPELLGGPSVFLFPPK |
| | | PKDTLMISRTPEVTCWVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPSREEMTKN |
| | | QVSLTCLVKGFYPSDIAVEWESNGQPENNYKTTPPVLDS |
| | | DGSFFLYSKLTVDKSRWQQGNVFSCSVMHEALHNHYT |
| | | QKSLSLSPGK |
| 42 | Ocrelizumab | EVQLVESGGGLVQPGGSLRLSCAASGYTFTSYNMHWVR |
| | heavy chain | QAPGKGLEWVGAIYPGNGDTSYNQKFKGRFTISVDKSK |
| | variable region | NTLYLQMNSLRAEDTAVYYCARVVYYSNSYWYFDVW |
| | | GQGTLVTVSSA |
| 43 | Ocrelizumab | GYTFTSY |
| | VH CDR1 | |
| 44 | Ocrelizumab | YPGNGD |
| | VH CDR2 | |
| 45 | Ocrelizumab | VVYYSNSYWYFDV |
| | VH CDR3 | |
| 46 | Ocrelizumab | DIQMTQSPSSLSASVGDRVTITCRASSSVSYMHWYQQKP |
| | light chain | GKAPKPLIYAP |
| | | SNLASGVPSRFSGSGSGTDFTLTISSLQPEDFATYYCQQW |
| | | SFNPPTFGQG |
| | | TKVEIKRTVAAPSVFIFPPSDEQLKSGTASWCLLNNFYPR |
| | | EAKVQWKVDN |
| | | ALQSGNSQESVTEQDSKDSTYSLSSTLTLSKADYEKHKV |
| | | YACEVTHQGLS |
| | | SPVTKSFNRGEC |
| 47 | Ocrelizumab | DIQMTQSPSSLSASVGDRVTITCRASSSVSYMHWYQQKP |
| | light chain | GKAPKPLIYAP |
| | variable region | SNLASGVPSRFSGSGSGTDFTLTISSLQPEDFATYYCQQW |
| | | SFNPPTFGQG |
| | | TKVEIKR |
| 48 | Ocrelizumab | SSVSYMH |
| | VL CDR1 | |
| 49 | Ocrelizumab | APSNLAS |
| | VL CDR2 | |
| 50 | Ocrelizumab | QQWSFNPPT |
| | VL CDR3 | |

In one embodiment, the antibody-EV conjugate comprises an antibody or antigen binding portion thereof comprising one, two, or three heavy chain CDR regions set forth in Table 6, or a sequence having at least 95%, 96%, 97%, 98%, or 99% identity thereto, and/or one, two, or three light chain CDR regions set forth in Table 6, or a sequence having at least 95%, 96%, 97%, 98%, or 99% identity thereto. In one embodiment, the antibody or antigen binding portion thereof comprises a VH CDR1 of SEQ ID NO:43, a VH CDR2 of SEQ ID NO:44, and a VH CDR3 of SEQ ID NO:45, and/or a VL CDR1 of SEQ ID NO:48, a VL CDR2 of SEQ ID NO:49, and a VL CDR3 of SEQ ID NO:50.

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:42, and/or a light chain variable region (LCVR) comprising at least 95%, at least 96%, at least 97%, at least 98%, at least 99% or 100% identity to the amino acid sequence set forth in SEQ ID NO:47.

In one embodiment, the antibody-EV conjugate comprises a heavy chain comprising at least 95%, at least 96%, at least 97%, at least 98%, at least 99% or 100% identity to the amino acid sequence set forth in SEQ ID NO:41, and/or a light chain comprising at least 95%, at least 96%, at least 97%, at least 98%, at least 99% or 100% identity to the amino acid sequence set forth in SEQ ID NO:46.

In another exemplary embodiment, the antibody-EV conjugates provided herein can comprise an antibody or antigen binding portion thereof that specifically binds to a human alpha-4 integrin protein. Antibody-EV conjugates comprising an antibody or antigen binding portion thereof that specifically binds to α-4 integrin can be used in the treatment of diseases or disorders in which α-4 integrin is implicated, for example, in the treatment of multiple sclerosis, e.g., relapsing-remitting multiple sclerosis. In an exemplary embodiment, the antibody-EV conjugates provided herein can comprise the anti-α-4 integrin antibody natalizumab, or an antigen binding portion thereof. Natalizumab is a humanized monoclonal antibody targeting α-4 integrin. Natalizumab is described in, e.g., U.S. Pat. Nos. 7,157,086, 6,602,503, and 8,124,350, each of which is incorporated by reference in its entirety.

In one embodiment, the antibody-EV conjugates described herein include an anti-α-4 integrin antibody or antigen binding portion thereof that comprises the variable heavy and/or light chain regions of natalizumab. In one embodiment, the α-4 integrin antibody comprises a heavy chain variable region comprising a VH CDR1, VH CDR2 and VH CDR3 of natalizumab, and/or a light chain variable region comprising a VL CDR1, VL CDR2 and VL CDR3 of natalizumab. The heavy chain and light chain amino acid sequences of natalizumab are described in Table 7.

TABLE 7
Amino Acid Sequence of Natalizumab

| SEQ | | |
|---|---|---|
| ID | SEQUENCE | |
| NO: | DESCRIPTION | AMINO ACID SEQUENCE |
| 51 | Natalizumab | VKLQQSGAELVKPGASVKLFCTASGFNIKDTY |
| | heavy chain | MHWVKQRPQQGLEWIGRIDPASGDTKYDPKFQ |
| | variable | VKATITADTSSNTAWLQLSSLTSEDTAVYYCA |
| | region | DGMWVSTGYALDFWGQGTTVTVSS |
| 52 | Natalizumab | GFNIKDT |
| | VH CDR1 | |
| 53 | Natalizumab | DPASGD |
| | VH CDR2 | |
| 54 | Natalizumab | GMWVSTGYALDF |
| | VH CDR3 | |
| 55 | Natalizumab | SIVMTQTPKFLLVSAGDRVTITCKASQSVTND |
| | light chain | VAWYQQKPGQSPKLLIYY |
| | variable | ASNRYTGVPDRFTGSGYGTDFTFTISTVQAED |
| | region | LAVYFCQQDYSSPYTFGG |
| | | GTKLEI |
| 56 | Natalizumab | QSVTNDVA |
| | VL CDR1 | |
| 57 | Natalizumab | YASNRYT |
| | VL CDR2 | |
| 58 | Natalizumab | QQDYSSPYT |
| | VL CDR3 | |

In one embodiment, the antibody-EV conjugate comprises an antibody or antigen binding portion thereof comprising one, two, or three heavy chain CDR regions set forth in Table 7, or a sequence having at least 95%, 96%, 97%, 98%, or 99% identity thereto, and/or one, two, or three light chain CDR regions set forth in Table 7, or a sequence having at least 95%, 96%, 97%, 98%, or 99% identity thereto. In one embodiment, the antibody or antigen binding portion thereof comprises a VH CDR1 of SEQ ID NO:52, a VH CDR2 of SEQ ID NO:53, and a VH CDR3 of SEQ ID NO:54, and/or a VL CDR1 of SEQ ID NO:56, a VL CDR2 of SEQ ID NO:57, and a VL CDR3 of SEQ ID NO:58.

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:51, and/or a light chain variable region (LCVR) comprising at least 95%, at least 96%, at least 97%, at least 98%, at least 99% or 100% identity to the amino acid sequence set forth in SEQ ID NO:55.

In another exemplary embodiment, the antibody-EV conjugates provided herein can comprise an antibody or antigen binding portion thereof that specifically binds to a human GD2 ganglioside (GD2) protein. Antibody-EV conjugates comprising an antibody or antigen binding portion thereof that specifically binds to GD2 can be used in the treatment of diseases or disorders in which GD2 is implicated, for example, in the treatment of neuroblastoma. In an exemplary embodiment, the antibody-EV conjugates provided herein can comprise the anti-GD2 antibody dinutuximab, or an antigen binding portion thereof. Dinutuximab is a chimeric human/mouse monoclonal antibody targeting GD2. Dinutuximab is described in, e.g., U.S. Pat. Nos. 9,777,068, 10,294,305, and 9,840,566, each of which is incorporated by reference in its entirety.

In one embodiment, the antibody-EV conjugates described herein include an anti-GD2 antibody or antigen binding portion thereof that comprises the variable heavy and/or light chain regions of dinutuximab. In one embodiment, the GD2 antibody comprises a heavy chain variable region comprising a VH CDR1, VH CDR2 and VH CDR3 of dinutuximab, and/or a light chain variable region comprising a VL CDR1, VL CDR2 and VL CDR3 of dinutuximab. The heavy chain and light chain amino acid sequences of dinutuximab are described in Table 8.

TABLE 8
Amino Acid Sequence of Dinutuximab

| SEQ | | |
|---|---|---|
| ID | SEQUENCE | |
| NO: | DESCRIPTION | AMINO ACID SEQUENCE |
| 59 | Dinutuximab | EVQLLQSGPELEKPGASVMISCKASGSSFTGYNMNWVR |
| | heavy chain | QNIGKSLEWIGA |
| | | IDPYYGGTSYNQKFKGRATLTVDKSSSTAYMHLKSLTSE |
| | | DSAVYYCVSGMEYWGQGTSVTVSSASTKGPSVFPLAPS |
| | | SKSTSGGTAALGCLVKDYFPEPVTVSWNSGALTSGVHT |
| | | FPAVLQSSGLYSLSSVVTVPSSSLGTQTYICNVNH |
| | | KPSNTKVDKRVEPKSCDKTHTCPPCPAPELLGGPSVFLFP |
| | | PKPKDTLMIS |
| | | RTPEVTCVVVDVSHEDPEVKFNWYVDGVEVHNAKTKP |
| | | REEQYNSTYRVVSVLTVLHQDWLNGKEYKCKVSNKAL |
| | | PAPIEKTISKAKGQPREPQVYTLPPSREEMTKNQVSLTCL |
| | | VKGFYPSDIAVEWESNGQPENNYKTTPPVLDSDGSFFLY |
| | | SKLTVDKSRWQQGNVFSCSVMHEALHNHYTQKSLSLSP |
| | | GK |
| 60 | Dinutuximab | EVQLLQSGPELEKPGASVMISCKASGSSFTGYNMNWVR |
| | heavy chain | QNIGKSLEWIGA |
| | variable region | IDPYYGGTSYNQKFKGRATLTVDKSSSTAYMHLKSLTSE |
| | | DSAVYYCVSGMEYWGQGTSVTVSSA |
| 61 | Dinutuximab | GSSFTGY |
| | VH CDR1 | |
| 62 | Dinutuximab | DPYYGG |
| | VH CDR2 | |
| 63 | Dinutuximab | GMEY |
| | VH CDR3 | |
| 64 | Dinutuximab | EIVMTQSPATLSVSPGERATLSCRSSQSLVHRNGNTYLH |
| | light chain | WYLQKPGQSPK |
| | | LLIHKVSNRFSGVPDRFSGSGSGTDFTLKISRVEAEDLGV |
| | | YFCSQSTHVP |
| | | PLTFGAGTKLELKRTVAAPSVFIFPPSDEQLKSGTASVVC |
| | | LLNNFYPREA |
| | | KVQWKVDNALQSGNSQESVTEQDSKDSTYSLSSTLTLS |
| | | KADYEKHKVYACEVTHQGLSSPVTKSFNRGEC |
| 65 | Dinutuximab | EIVMTQSPATLSVSPGERATLSCRSSQSLVHRNGNTYLH |
| | light chain | WYLQKPGQSPK |
| | variable region | LLIHKVSNRFSGVPDRFSGSGSGTDFTLKISRVEAEDLGV |
| | | YFCSQSTHVP |
| | | PLTFGAGTKLELKR |
| 66 | Dinutuximab | QSLVHRNGNTYLH |
| | VL CDR1 | |
| 67 | Dinutuximab | KVSNRFS |
| | VL CDR2 | |
| 68 | Dinutuximab | SQSTHVPPLT |
| | VL CDR3 | |

In one embodiment, the antibody-EV conjugate comprises an antibody or antigen binding portion thereof comprising one, two, or three heavy chain CDR regions set forth in Table 8, or a sequence having at least 95%, 96%, 97%, 98%, or 99% identity thereto, and/or one, two, or three light chain CDR regions set forth in Table 8, or a sequence having at least 95%, 96%, 97%, 98%, or 99% identity thereto. In one embodiment, the antibody or antigen binding portion thereof comprises a VH CDR1 of SEQ ID NO:61, a VH CDR2 of SEQ ID NO:62, and a VH CDR3 of SEQ ID NO:63, and/or a VL CDR1 of SEQ ID NO:66, a VL CDR2 of SEQ ID NO:67, and a VL CDR3 of SEQ ID NO:68.

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:60, and/or a light chain variable region (LCVR) comprising at least 95%, at least 96%, at least 97%, at least 98%, at least 99% or 100% identity to the amino acid sequence set forth in SEQ ID NO:65.

In one embodiment, the antibody-EV conjugate comprises a heavy chain comprising at least 95%, at least 96%, at least 97%, at least 98%, at least 99% or 100% identity to the amino acid sequence set forth in SEQ ID NO:59, and/or a light chain comprising at least 95%, at least 96%, at least 97%, at least 98%, at least 99% or 100% identity to the amino acid sequence set forth in SEQ ID NO:64.

In another exemplary embodiment, the antibody-EV conjugates provided herein can comprise an antibody or antigen binding portion thereof that specifically binds to a human amyloid beta protein, associated with Alzheimer's Disease. Antibody-EV conjugates comprising an antibody or antigen binding portion thereof that specifically binds to amyloid beta can be used in the treatment of diseases or disorders in which amyloid beta is implicated, for example, in the treatment of Alzheimer's Disease. In an exemplary embodiment, the antibody-EV conjugates provided herein can comprise the anti-amyloid beta antibody gantenerumab, or an antigen binding portion thereof. Gantenerumab is a human IgG1 monoclonal antibody targeting amyloid beta. Gantenerumab is described in, e.g., U.S. Pat. Nos. 7,794,719, 8,216,577, and 8,329,886, each of which is incorporated by reference in its entirety.

In one embodiment, the antibody-EV conjugates described herein include an anti-amyloid beta antibody or antigen binding portion thereof that comprises the variable heavy and/or light chain regions of gantenerumab. In one embodiment, the amyloid beta antibody comprises a heavy chain variable region comprising a VH CDR1, VH CDR2 and VH CDR3 of gantenerumab, and/or a light chain variable region comprising a VL CDR1, VL CDR2 and VL CDR3 of gantenerumab. The heavy chain and light chain amino acid sequences of gantenerumab are described in Table 9.

TABLE 9
Amino Acid Sequence of Gantenerumab

| SEQ | | |
|---|---|---|
| ID | SEQUENCE | |
| NO: | DESCRIPTION | AMINO ACID SEQUENCE |
| 69 | Gantenerumab | QVELVESGGGLVQPGGSLRLSCAASGFTFSSYAMSWVR |
| | heavy chain | QAPGKGLEWVSAINASGTRTYYADSVKGRFTISRDNSK |
| | | NTLYLQMNSLRAEDTAVYYCARGKGNTHKPYGYVRYF |
| | | DVWGQGTLVTVSSASTKGPSVFPLAPSSKSTSGGTAALG |
| | | CLVKDYFPEPVTVSWNSGALTSGVHTFPAVLQSSGLYSL |
| | | SSVVTVPSS |
| | | SLGTQTYICNVNHKPSNTKVDKKVEPKSCDKTHTCPPCP |
| | | APELLGGPSVF |
| | | LFPPKPKDTLMISRTPEVTCVVVDVSHEDPEVKFNWYV |
| | | DGVEVHNAKTKP |
| | | REEQYNSTYRVVSVLTVLHQDWLNGKEYKCKVSNKAL |
| | | PAPIEKTISKAKG |
| | | QPREPQVYTLPPSRDELTKNQVSLTCLVKGFYPSDIAVE |
| | | WESNGQPENNY |
| | | KTTPPVLDSDGSFFLYSKLTVDKSRWQQGNVFSCSVMH |
| | | EALHNHYTQKSLSLSPGK |
| 70 | Gantenerumab | QVELVESGGGLVQPGGSLRLSCAASGFTFSSYAMSWVR |
| | heavy chain | QAPGKGLEWVSAINASGTRTYYADSVKGRFTISRDNSK |
| | variable region | NTLYLQMNSLRAEDTAVYYCARGKGNTHKPYGYVRYF |
| | | DVWGQGTLVTVSSA |
| 71 | Gantenerumab | GFTFSSY |
| | VH CDR1 | |
| 72 | Gantenerumab | NASGTR |
| | VH CDR2 | |
| 73 | Gantenerumab | GKGNTHKPYGYVRYFDV |
| | VH CDR3 | |
| 74 | Gantenerumab | DIVLTQSPATLSLSPGERATLSCRASQSVSSSYLAWYQQ |
| | light chain | KPGQAPRLLIY |
| | | GASSRATGVPARFSGSGSGTDFTLTISSLEPEDFATYYCL |
| | | QIYNMPITFG |
| | | QGTKVEIKRTVAAPSVFIFPPSDEQLKSGTASVVCLLNNF |
| | | YPREAKVQWK |
| | | VDNALQSGNSQESVTEQDSKDSTYSLSSTLTLSKADYEK |
| | | HKVYACEVTHQ |
| | | GLSSPVTKSFNRGEC |
| 75 | Gantenerumab | DIVLTQSPATLSLSPGERATLSCRASQSVSSSYLAWYQQ |
| | light chain | KPGQAPRLLIY |
| | variable region | GASSRATGVPARFSGSGSGTDFTLTISSLEPEDFATYYCL |
| | | QIYNMPITFG |
| | | QGTKVEIKR |
| 76 | Gantenerumab | QSVSSSYLA |
| | VL CDR1 | |
| 77 | Gantenerumab | GASSRAT |
| | VL CDR2 | |
| 78 | Gantenerumab | LQIYNMPIT |
| | VL CDR3 | |

In one embodiment, the antibody-EV conjugate comprises an antibody or antigen binding portion thereof comprising one, two, or three heavy chain CDR regions set forth in Table 9, or a sequence having at least 95%, 96%, 97%, 98%, or 99% identity thereto, and/or one, two, or three light chain CDR regions set forth in Table 9, or a sequence having at least 95%, 96%, 97%, 98%, or 99% identity thereto. In one embodiment, the antibody or antigen binding portion thereof comprises a VH CDR1 of SEQ ID NO:71, a VH CDR2 of SEQ ID NO:72, and a VH CDR3 of SEQ ID NO:73, and/or a VL CDR1 of SEQ ID NO:76, a VL CDR2 of SEQ ID NO:77, and a VL CDR3 of SEQ ID NO:78.

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:70, and/or a light chain variable region (LCVR) comprising at least 95%, at least 96%, at least 97%, at least 98%, at least 99% or 100% identity to the amino acid sequence set forth in SEQ ID NO:75.

In one embodiment, the antibody-EV conjugate comprises a heavy chain comprising at least 95%, at least 96%, at least 97%, at least 98%, at least 99% or 100% identity to the amino acid sequence set forth in SEQ ID NO:69, and/or a light chain comprising at least 95%, at least 96%, at least 97%, at least 98%, at least 99% or 100% identity to the amino acid sequence set forth in SEQ ID NO:74.

In another exemplary embodiment, the antibody-EV conjugates provided herein can comprise the anti-amyloid beta antibody lecanemab, or an antigen binding portion thereof. Lecanemab is a humanized mouse monoclonal antibody targeting amyloid beta. Lecanemab is described in, e.g., U.S. Pat. Nos. 8,106,164, 8,999,936, and 9,573,994, each of which is incorporated by reference in its entirety.

In one embodiment, the antibody-EV conjugates described herein include an anti-amyloid beta antibody or antigen binding portion thereof that comprises the variable heavy and/or light chain regions of lecanemab. In one embodiment, the amyloid beta antibody comprises a heavy chain variable region comprising a VH CDR1, VH CDR2 and VH CDR3 of lecanemab, and/or a light chain variable region comprising a VL CDR1, VL CDR2 and VL CDR3 of lecanemab. The heavy chain and light chain amino acid sequences of lecanemab are described in Table 10.

TABLE 10
Amino Acid Sequence of Lecanemab

| SEQ | | |
|---|---|---|
| ID | SEQUENCE | |
| NO: | DESCRIPTION | AMINO ACID SEQUENCE |
| 79 | Lecanemab | EVQLVESGGGLVQPGGSLRLSCSASGFTFSSFGMHWVR |
| | heavy chain | QAPGKGLEWVAYISSGSSTIYYGDTVKGRFTISRDNAKN |
| | | SLFLQMSSLRAEDTAVYYCAREG |
| | | GYYYGRSYYTMDYWGQGTTVTVSSASTKGPSVFPLAPS |
| | | SKSTSGGTAALGCLVKDYFPEPVTVSWNSGALTSGVHT |
| | | FPAVLQSSGLYSLSSVVTVPSSSL |
| | | GTQTYICNVNHKPSNTKVDKRVEPKSCDKTHTCPPCPAP |
| | | ELLGGPSVFLF |
| | | PPKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVYTLPPSREE |
| | | MTKNQVSLTCLVKGFYPSDIAVEWESNGQPENNYKTTP |
| | | PVLDSDGSFFLYSKLTVDKSRWQQGNVFSCSVMHEALH |
| | | NHYTQKSLSLSPGK |
| 80 | Lecanemab | EVQLVESGGGLVQPGGSLRLSCSASGFTFSSFGMHWVR |
| | heavy chain | QAPGKGLEWVAYISSGSSTIYYGDTVKGRFTISRDNAKN |
| | variable region | SLFLQMSSLRAEDTAVYYCAREG |
| | | GYYYGRSYYTMDYWGQGTTVTVSSA |
| 81 | Lecanemab | DVVMTQSPLSLPVTPGAPASISCRSSQSIVHSNGNTYLEW |
| | light chain | YLQKPGQSPK |
| | | LLIYKVSNRFSGVPDRFSGSGSGTDFTLRISRVEAEDVGI |
| | | YYCFQGSHVPPTFGPGTKLEIKRTVAAPSVFIFPPSDEQL |
| | | KSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| 82 | Lecanemab | DVVMTQSPLSLPVTPGAPASISCRSSQSIVHSNGNTYLEW |
| | light chain | YLQKPGQSPKLLIYKVSNRFSGVPDRFSGSGSGTDFTLRI |
| | variable region | SRVEAEDVGIYYCFQGSHVPPTFGPGTKLEIKR |

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:80, and/or a light chain variable region (LCVR) comprising at least 95%, at least 96%, at least 97%, at least 98%, at least 99% or 100% identity to the amino acid sequence set forth in SEQ ID NO:82.

In one embodiment, the antibody-EV conjugate comprises a heavy chain comprising at least 95%, at least 96%, at least 97%, at least 98%, at least 99% or 100% identity to the amino acid sequence set forth in SEQ ID NO:79, and/or a light chain comprising at least 95%, at least 96%, at least 97%, at least 98%, at least 99% or 100% identity to the amino acid sequence set forth in SEQ ID NO:81.

In another exemplary embodiment, the antibody-EV conjugates provided herein can comprise an antibody or antigen binding portion thereof that specifically binds to a human B-lymphocyte antigen CD20 (CD20) protein. As noted previously, antibody-EV conjugates comprising an antibody or antigen binding portion thereof that specifically binds to CD20 can be used in the treatment of diseases or disorders in which CD20 is implicated, for example, in the treatment of multiple sclerosis, e.g., relapsing multiple sclerosis. In an exemplary embodiment, the antibody-EV conjugates provided herein can comprise the anti-CD20 antibody ublituximab, or an antigen binding portion thereof. Ublituximab is a chimeric human/mouse monoclonal antibody targeting CD20. Ublituximab is described in, e.g., U.S. Pat. Nos. 9,234,045, 9,694,071, and 9,873,745, each of which is incorporated by reference in its entirety.

In one embodiment, the antibody-EV conjugates described herein include an anti-CD20 antibody or antigen binding portion thereof that comprises the variable heavy and/or light chain regions of ublituximab. In one embodiment, the CD20 antibody comprises a heavy chain variable region comprising a VH CDR1, VH CDR2 and VH CDR3 of ublituximab, and/or a light chain variable region comprising a VL CDR1, VL CDR2 and VL CDR3 of ublituximab. The heavy chain and light chain amino acid sequences of ublituximab are described in Table 11.

TABLE 11
Amino Acid Sequence of Ublituximab

| SEQ | | |
|---|---|---|
| ID | SEQUENCE | |
| NO: | DESCRIPTION | AMINO ACID SEQUENCE |
| 83 | Ublituximab | QAYLQQSGAELVRPGASVKMSCKASGYTFTSYNMHWV |
| | heavy chain | KQTPRQGLEWIGGIYPGNGDTSYNQKFKGKATLTVGKS |
| | | SSTAYMQLSSLTSEDSAVYFCARYDYNYAMDYWGQGT |
| | | SVTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSRDELTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| 84 | Ublituximab | QAYLQQSGAELVRPGASVKMSCKASGYTFTSYNMHWV |
| | heavy chain | KQTPRQGLEWIGGIYPGNGDTSYNQKFKGKATLTVGKS |
| | variable region | SSTAYMQLSSLTSEDSAVYFCARYDYNYAMDYWGQGT |
| | | SVTVSSA |
| 85 | Ublituximab | GYTFTSY |
| | VH CDR1 | |
| 86 | Ublituximab | YPGNGD |
| | VH CDR2 | |
| 87 | Ublituximab | YDYNYAMDY |
| | VH CDR3 | |
| 88 | Ublituximab | QIVLSQSPAILSASPGEKVTMTCRASSSVSYMHWYQQKP |
| | light chain | GSSPKPWIYAT |
| | | SNLASGVPARFSGSGSGTSYSFTISRVEAEDAATYYCQQ |
| | | WTFNPPTFGGG |
| | | TRLEIKRTVAAPSVFIFPPSDEQLKSGTASVVCLLNNFYP |
| | | REAKVQWKVD |
| | | NALQSGNSQESVTEQDSKDSTYSLSSTLTLSKADYEKHK |
| | | VYACEVTHQGL |
| | | SSPVTKSFNRGEC |
| 89 | Ublituximab | QIVLSQSPAILSASPGEKVTMTCRASSSVSYMHWYQQKP |
| | light chain | GSSPKPWIYAT |
| | variable region | SNLASGVPARFSGSGSGTSYSFTISRVEAEDAATYYCQQ |
| | | WTFNPPTFGGG |
| | | TRLEIKR |
| 90 | Ublituximab | SSVSYMH |
| | VL CDR1 | |
| 91 | Ublituximab | ATSNLAS |
| | VL CDR2 | |
| 92 | Ublituximab | QQWTFNPPT |
| | VL CDR3 | |

In one embodiment, the antibody-EV conjugate comprises an antibody or antigen binding portion thereof comprising one, two, or three heavy chain CDR regions set forth in Table 11, or a sequence having at least 95%, 96%, 97%, 98%, or 99% identity thereto, and/or one, two, or three light chain CDR regions set forth in Table 11, or a sequence having at least 95%, 96%, 97%, 98%, or 99% identity thereto. In one embodiment, the antibody or antigen binding portion thereof comprises a VH CDR1 of SEQ ID NO:85, a VH CDR2 of SEQ ID NO:86, and a VH CDR3 of SEQ ID NO:87, and/or a VL CDR1 of SEQ ID NO:90, a VL CDR2 of SEQ ID NO:91, and a VL CDR3 of SEQ ID NO:92.

In one embodiment, the antibody-EV conjugate comprises a heavy chain variable region (HCVR) comprising at least 95%, at least 96%, at least 97%, at least 98%, at least 99% or 100% identity to the amino acid sequence set forth in SEQ ID NO:84, and/or a light chain variable region (LCVR) comprising at least 95%, at least 96%, at least 97%, at least 98%, at least 99% or 100% identity to the amino acid sequence set forth in SEQ ID NO:89.

In one embodiment, the antibody-EV conjugate comprises a heavy chain comprising at least 95%, at least 96%, at least 97%, at least 98%, at least 99% or 100% identity to the amino acid sequence set forth in SEQ ID NO:83, and/or a light chain comprising at least 95%, at least 96%, at least 97%, at least 98%, at least 99% or 100% identity to the amino acid sequence set forth in SEQ ID NO:88.

In one embodiment, the antibody-EV conjugates described herein include human immune globulin. Immune globulin is a sterilized solution made from human plasma that contains human antibodies. Immune globulin products are sold under the trade names GAMUNEX C™, GAMMAKED™, HIZENTRA™, PRIVIGEN™, and GAMMAGARD™. Antibody-EV conjugates comprising immune globulin can be used in the treatment of disorders including, but not limited to, chronic inflammatory demyelinating polyneuropathy (CIDP) and multifocal motor neuropathy.

In some embodiments, the antibody-labeled EVs provided herein can additionally comprise molecules including but not limited to small molecules, nucleic acid (e.g., short interfering RNAs (siRNAs), short hairpin RNAs (shRNA), micro RNAs (miRNAs) and double-stranded RNAs (dsRNA)), protein and/or peptide.

In some instances, EVs are isolated from cells and subsequently loaded with the desired molecules prior to the administration of the EVs into a subject. In other instances, the desired molecules can be loaded into the EVs by co-incubation of the cell producing EVs with one or more therapeutic agents. In some other instances, a desired nucleic acid, protein or peptide can be loaded into the EVs by overexpression of the desired molecule in the cell which is used to produce the EVs, so that the EVs are loaded with the desired molecule during production. In yet another instance, the desired molecules can be loaded into the EVs by overexpression of a carrier molecule (e.g., protein) that facilitates trafficking of the desired molecule to exosomes. For example, a carrier peptide from Bovine Leukemia Virus Protein can be expressed as a fusion protein with a polypeptide of interest, to facilitate loading of the polypeptide of interest into exosomes. This approach is described, for example, in U.S. Pat. No. 9,546,371, the entire contents of which are incorporated herein by reference. In other embodiments, a carrier molecule is expressed with an inhibitory nucleic acid in the cell which is used to produce the EVs, so that the EVs can be loaded with the inhibitory nucleic acid.

In the instances described above, the desired molecules can be exogenous or endogenous. Exogenous molecules refer to molecules that are added to EVs from an external source, e.g., not natural to the EVs, in vitro, in vivo or ex vivo. Endogenous molecules refer to molecules that are natively present inside or associated with EVs in vitro, in vivo or ex vivo. Exogenous can molecules can include the same type of molecule (nucleic acid, protein, etc.) that is natively present in EVs, when the molecule is added to the EVs from an external source, e.g., by overexpression, post-production loading, etc.

EVs can be loaded with desired cargo molecules prior to or after surface labeling the EVs with an antibody, or antigen binding portion thereof. Desired molecules described herein can be introduced into the EVs by a number of different techniques including incubation, sonication, electroporation or the use of a transfection reagent such as membrane permeabilizers (e.g., polyols, detergents, sugars). Electroporation conditions may vary depending on the charge and size of the desired molecule. Typical voltages are in the range of 20V/cm to 1,000V/cm, such as 20V/cm to 100V/cm with capacitance typically between 25 μF and 250 μF, such as between 25 μF and 125 μF. A voltage in the range of 150 mV to 250 mV, particularly a voltage of 200 mV is preferred for loading EVs with an antibody. Alternatively, the EVs may be loaded with desired molecules (e.g., exogenous protein and/or peptide) using a transfection reagent. Despite the small size of the EVs, conventional transfection agents may be used for transfection of EVs with desired molecules (e.g., exogenous protein and/or peptide). In one instance, EVs may also be loaded by transforming or transfecting a host cell with a nucleic acid construct which expresses a therapeutic protein or peptide of interest, such that the therapeutic protein or peptide is taken up into the EVs as the EVs are produced from the cell. In another instance, EVs may also be loaded with an inhibitory nucleic acid construct by transforming or transfecting a host cell with the inhibitory nucleic acid construct with or without a carrier/adapter molecule, such that the inhibitory nucleic acid construct is taken up into the EVs.

(iii) EVs Loaded with Antibodies in the EV Lumen

In one aspect, the invention provides a composition comprising EVs loaded with a polypeptide (e.g., antibody, or antigen-binding fragment thereof) in the lumen of the EV. The EVs suitable for this purpose can comprise an extracellular vesicle derived from neural cells (e.g., neural progenitor cells or neural stem cells) for loading with an antibody, or antigen-binding portion thereof. Any of the antibodies described herein can be used to load the EVs in the EV lumen, in addition to or alternatively to loading on the EV surface, e.g., via click chemistry, as described above.

A polypeptide (e.g., antibody or antigen binding portion thereof) can be loaded into the lumen of the EV using a variety of methods.

In some embodiments, the antibody is loaded into the vesicle using a detergent suitable for membrane permeabilization, e.g., saponin. In some embodiments, the antibody is loaded into the vesicle by sonication. In some embodiments, the antibody is loaded into the vesicle by electroporation. In some embodiments, the antibody is loaded into the vesicle by incubation (e.g., at room temperature) with shaking (e.g., 100 rpm-1500 rpm). In some embodiments, the antibody is loaded into the vesicle using a combination of the foregoing methods (e.g., saponin permeabilization and sonication, electroporation and sonication, saponin permeabilization and electroporation, etc.). Protein, e.g., antibody, can be added to the vesicles prior to, during, or after permeabilization, sonication, and/or electroporation. In some embodiments, the vesicles are permeabilized using detergents, sonication, and/or electroporation in the presence of the protein to be loaded into the lumen, e.g., antibody. In other embodiments, the vesicles are permeabilized using detergents, sonication, and/or electroporation, and are subsequently incubated with the protein to be loaded, e.g., antibody. In some embodiments, the protein (e.g., antibody) to be loaded is added to the vesicles at a concentration of about 25 to about 2000 μg/mL. In some embodiments, the protein (e.g., antibody) to be loaded is added to the vesicles at a concentration of about 50 to about 1500 μg/mL. In some embodiments, the protein (e.g., antibody) to be loaded is added to the vesicles at a concentration of about 100 to about 1000 μg/mL. In some embodiments, the protein (e.g., antibody) to be loaded is added to the vesicles at a concentration of about 25 ug/mL, about 50 ug/mL, about 100 ug/mL, about 200 ug/mL, about 300 ug/mL, about 400 ug/mL, about 500 ug/mL, about 600 ug/mL, about 700 ug/mL, about 800 ug/mL, about 900 ug/mL, about 1,000 ug/mL, about 1,500 ug/mL, or about 2000 ug/mL.

In some embodiments, the antibody is loaded into the lumen of the vesicle using a detergent suitable for membrane permeabilization. Such detergents include, e.g., saponin, Tween-20, and other detergents known in the art suitable for extracellular vesicle permeabilization. In some embodiments, about 0.01%, about 0.03%, about 0.05%, about 0.07%, about 0.09%, about 0.1%, about 0.12%, about 0.14%, about 0.16%, about 0.18%, about 0.2%, about 0.22%, about 0.24%, about 0.26%, about 0.28%, or about 0.3% (w/v) of the detergent (e.g., saponin) is used to permeabilize the EV membrane. In some embodiments, the antibody is loaded into the lumen of EVs by permeabilization of the EV membrane using saponin. For example, the membrane can be permeabilized by incubating a preparation of EVs with, e.g., 0.01%-5% (w/v) saponin, prior to addition of the antibody. In some embodiments, the membrane can be permeabilized by incubating a preparation of EVs with, e.g., about 0.01%, about 0.03%, about 0.05%, about 0.07%, about 0.09%, about 0.1%, about 0.12%, about 0.14%, about 0.16%, about 0.18%, about 0.2%, about 0.22%, about 0.24%, about 0.26%, about 0.28%, about 0.3%, about 0.4%, about 0.5%, about 0.6%, about 0.7%, about 0.8%, about 0.9%, about 1.0%, about 1.2%, about 1.4%, about 1.6%, about 1.8%, about 2.0%, about 2.2%, about 2.4%, about 2.8%, about 3.0%, about 3.2%, about 3.4%, about 3.6%, about 3.8%, about 4.0%, about 4.2%, about 4.4%, about 4.6%, about 4.8%, or about 5.0% (w/v) saponin, prior to addition of the antibody. In some embodiments, the EVs can be incubated with saponin for 1-20 minutes prior to the addition of the antibody. In some embodiments, the EVs can be incubated with saponin for 1-10 minutes prior to the addition of the antibody. In some embodiments, the EVs can be incubated with saponin for 1-5 minutes prior to the addition of the antibody. The antibody can then be added to the permeabilized EVs and incubated, optionally with gentle mixing or shaking (e.g., 100 rpm, 200 rpm, 300 rpm, 400 rpm, 500 rpm, 600 rpm, 700, rpm, 800 rpm, 900 rpm, 1000 rpm, 1100 rpm, 1200 rpm, 1300, rpm, 1400 rpm, 1500 rpm), for a duration of 1-30 minutes (e.g., 1-20 minutes, 1-15 minutes, 1-10 minutes, or 1-5 minutes).

In some embodiments, the antibody is loaded into the vesicle by sonication. Those skilled in the art will appreciate that sonication can be performed by a combination of differing amplitude, pulse time, and cycles, as exemplified in Table 12, herein. For example, the amplitude for sonication can be set anywhere from 10% to 100% amplitude (e.g., 10%, 20%, 30%, 40%, 50%, 60%, 70%, 80%, 90%, or 100%), combined with varying pulse times (e.g., 4 seconds on/2 seconds off; 4 seconds on/4 seconds off; 4 seconds on/8 seconds off; 2 seconds on/2 seconds off; 2 seconds on/4 seconds off; 2 seconds on/8 seconds off; 8 seconds on/2 seconds off; 8 seconds on/4 seconds off; 8 seconds on/8 seconds off) and number of cycles (e.g., 2 cycles to 36 cycles, e.g., 2 cycles, 4 cycles, 6 cycles, 8 cycles, 10 cycles, 12 cycles, 14 cycles, 16 cycles, 18 cycles, 20 cycles, 22 cycles, 24 cycles, 26 cycles, 28 cycles, 30 cycles, 32 cycles, 34 cycles, or 36 cycles). In some embodiments, the amplitude for sonication can be set anywhere from 10% to 100% amplitude (e.g., 10%, 20%, 30%, 40%, 50%, 60%, 70%, 80%, 90%, or 100%) at a constant duration (e.g., 1 minute, 2 minutes, 3 minutes, 4 minutes, 5 minutes, 6 minutes, 7 minutes, 8 minutes, 9 minutes, 10 minutes, or more). In some embodiments, the antibody is loaded into the vesicle by electroporation using known methods, e.g., by varying the voltage (e.g., 100V, 150V, 200V, 250V, 300V, 350V, 400V, 450V, 500V, 550V, 600V, 650V, 700V, 750V, 800V, 850V, 900V, 950V, 1 kV, or more) and the duration and number of pulses (e.g., 1 pulse, 2 pulses, 3 pulses, 4 pulses, 5 pulses, 6 pulses, 7 pulses, 8 pulses, 9 pulses, 10 pulses, 11 pulses, 12 pulses, 13 pulses, 14 pulses, 15 pulses, 16 pulses, 17 pulses, 18 pulses, 19 pulses, 20 pulses, or more).

In some embodiments, the antibody is loaded into the vesicle by incubation (e.g., for 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more) with or without shaking (e.g., 100 rpm, 200 rpm, 300 rpm, 400 rpm, 500 rpm, 600 rpm, 700, rpm, 800 rpm, 900 rpm, 1000 rpm, 1100 rpm, 1200 rpm, 1300, rpm, 1400 rpm, 1500 rpm). The incubation can be performed at any suitable temperature. In some embodiments, the incubation can be performed at about 4 to about 37° C. In some embodiments, the incubation can be performed at room temperature, on ice, at about 4° C., or at about 37° C.

In some embodiments, the polypeptide (e.g., antibody) is loaded into the vesicle using a combination of any of the methods described herein for introducing the antibody into the lumen of a vesicle, as also exemplified herein. In some embodiments, the polypeptide (e.g., antibody) is included (added to the vesicle) during vesicle membrane permeabilization. In some embodiments, the polypeptide (e.g. antibody) is included (added to the vesicle) after vesicle membrane permeabilization.

In certain embodiments, the antibody is loaded into the EVs by saponin permeabilization (e.g., using 0.01-5% saponin) of the EVs, with sonication at 10-100% amplitude, for a duration of 5-30 minutes, prior to adding the antibody to the EVs. In certain embodiments, the antibody is loaded into the EVs by saponin permeabilization (0.2% saponin) of the EVs, with sonication at 20% amplitude, for a duration of 10 minutes, prior to adding the antibody to the EVs. In certain embodiments, the antibody is loaded into the EVs by saponin permeabilization (0.2% saponin) of the EVs, with sonication at 40% amplitude, for a duration of 10 minutes, prior to adding the antibody to the EVs. In certain embodiments, the antibody is loaded into the EVs by saponin permeabilization (0.2% saponin) of the EVs, with sonication at 20% amplitude, for a duration of 5 minutes, prior to adding the antibody to the EVs.

In some embodiments, the antibody is loaded into the EVs by 0.01-5% saponin permeabilization combined with shaking. In some embodiments, the antibody is loaded into the EVs by 0.2% saponin permeabilization combined with shaking (e.g., 500 rpm). In some embodiments, the antibody is loaded into the EVs by incubation at room temperature with shaking (e.g., 1000 rpm). In some embodiments, the antibody is loaded into the EVs by electroporation. In some embodiments, the antibody is loaded into the vesicle by sonication at 20% amplitude, 12 cycles, 4 seconds on and 8 seconds off.

In some embodiments, the antibody is loaded in the EVs by sonication at 60% amplitude, for a duration of 4 seconds on/8 seconds off, for 6 cycles. In some embodiments, the antibody is loaded in the EVs by sonication at 60% amplitude, for a duration of 2 seconds on/4 seconds off, followed by incubation on ice for 2 minutes, for 6 cycles.

In some embodiments, a range of polypeptide (e.g., antibody) concentration can be loaded into the lumen of the EVs, e.g., 100 ug/ml, about 200 ug/ml, about 300 ug/ml, about 400 ug/ml, about 500 ug/ml, about 600 ug/ml, about 700 ug/ml, about 800 ug/ml, about 900 ug/ml, or about 1,000 ug/ml.

Upon loading of the antibody into the lumen of the EVs, the luminally-loaded EVs can be separated from the free antibodies and non-loaded EVs using various methods known in the art, including ultrafiltration, ultracentrifugation, and high performance liquid chromatography (HPLC), including size exclusion, ion exchange, and bioaffinity chromatography).

### C. Formulation, Delivery, and Administration

The polypeptide loaded EVs, e.g., antibody loaded EVs—including antibody-EV conjugates and EVs lumenally loaded with antibody—provided herein can be formulated in a pharmaceutical composition (e.g., a pharmaceutical composition comprising neural cell derived Ab-EV conjugates) for delivery to a subject. Pharmaceutical compositions can comprise a therapeutically effective amount of the polypeptide loaded EVs (e.g., antibody loaded neural EVs) and a pharmaceutically acceptable carrier. For example, a therapeutically effective amount of the polypeptide loaded EVs (e.g., Ab-EV conjugates and/or lumenally loaded EVs) can be provided in sterile phosphate-buffered saline. Other suitable excipients, vehicles, and carriers are known in the art, and are described, for example, in Remington's Pharmaceutical Sciences, 18th ed. (1990). It will be understood by those skilled in the art that any mode of administration, vehicle or carrier conventionally employed and which is inert with respect to the active agent may be utilized for preparing and administering pharmaceutical compositions comprising polypeptide loaded EVs of the present disclosure. Illustrative of such methods, vehicles and carriers are those described, for example, in Remington's Pharmaceutical Sciences, 4th ed. (1970), the disclosure of which is incorporated herein by reference.

Formulations containing the disclosed polypeptide loaded EVs (e.g., antibody loaded neural EVs) may take the form of a liquid, solid or semi-solid, such as, for example, solutions, suspensions, emulsions, sustained-release formulations, lotions, aerosols, or the like, optionally in unit dosage forms suitable for simple administration of precise dosages. Pharmaceutical compositions typically include a conventional pharmaceutical carrier and/or excipient and may additionally include other medicinal agents, carriers, adjuvants, additives and the like. The weight percentage ratio of the Ab-EV conjugates to the one or more excipients can be between about 20:1 to about 1:60, or between about 15:1 to about 1:45, or between about 10:1 to about 1:40, or between about 9:1, 8:1, 7:1, 6:1, 5:1, 4:1, 3:1, 2:1 or 1:1 to about 1:2, 1:3, 1:4, 1:5, 1:6, 1:7, 1:8, 1:9, 1:10, 1:15, 1:20, 1:25, 1:30, or 1:35, and preferably is about 20:1, 19:1, 18:1, 17:1, 16:1, 15:1, 14:1, 13:1, 12:1, 11:1, 10:1, 9:1, 8:1, 7:1, 6:1 or 5:1. In some embodiments, the disclosed composition comprises between about 1 μg to about 1 g or more of total EVs, for example, about 1 μg to about 100 μg, about 100 μg to about 200 μg, about 200 μg to about 300 μg, about 300 μg to about 400 μg, about 500 μg to about 600 μg, about 700 μg to about 800 μg, about 900 μg to about 1 mg, about 100 μg to about 500 μg, about 1 mg to about 500 mg, about 5 mg to about 500 mg, about 10 mg to about 500 mg, about 25 mg to about 500 mg, about 50 mg to about 350 mg, about 75 mg to about 450 mg, about 50 mg to about 450 mg, about 75 mg to about 325 mg, about 100 mg to about 650 mg, or about 500 mg to about 1 g of total EVs, and may optionally contain one or more suitable pharmaceutical carriers, additives and/or excipients.

In various instances, the pharmaceutical compositions described herein (e.g., pharmaceutical composition comprising polypeptide loaded EVs such as antibody loaded neural EVs) can be formulated for delivery to a cell and/or to a subject via any route of administration know to a person of skill in the art. Modes of administration are commonly known or are apparent to those skilled in the art; for example, see Remington's Pharmaceutical Sciences (17th Ed., Mack Pub. Co. 1985).

Compositions comprising the polypeptide loaded EVs (e.g., antibody loaded neural EVs) provided herein can delivered to a subject by any suitable route, including but not limited to injection, infusion, inhalation, intranasal, intraocular, topical delivery, intercannular delivery, or ingestion. Injection includes, without limitation, intravenous, intracranial, intrathecal, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some instances, administration includes intracerebrospinal injection. In some other instances, administration includes aerosol inhalation, e.g., with nebulization. In other instances, administration is systemic (e.g., oral, rectal, nasal, sublingual, buccal, or parenteral), enteral (e.g., system-wide effect, but delivered through the CNS), or local (e.g., local application on the skin, intravitreal injection). In some instances, administration of the polypeptide loaded EVs (e.g., antibody loaded neural EVs), is capable of delivering the EVs loaded with an antibody to the central nervous system, and in particular embodiments, across the blood-brain barrier. In some embodiments, administration is at the site of diseased and/or dysfunctional tissue (e.g., brain). In other embodiments, the site of administration is distal to the site of diseased and/or dysfunctional tissue (e.g., in the case of intravenous or intranasal delivery). In one embodiment, a composition comprising the polypeptide loaded EVs is administered to a subject parenterally. In one embodiment, a composition comprising the polypeptide loaded EVs is administered to a subject intravenously. In another embodiment, a composition comprising the polypeptide loaded EVs is administered to a subject intranasally. In one embodiment, a composition comprising the polypeptide loaded EVs is administered to a subject intracranially. In one embodiment, a composition comprising the polypeptide loaded EVs is administered to a subject intrathecally.

An injectable composition for parenteral administration (e.g. intravenous, intramuscular, intrathecal, intracerebrospinal fluid, or intranasal), can contain the polypeptide loaded EVs (e.g., antibody loaded neural EVs) and optionally additional components in a suitable i.v. solution, such as sterile physiological salt solution. In other embodiments, the composition is formulated as a suspension in an aqueous emulsion.

Liquid pharmaceutical compositions can be prepared by dissolving or dispersing a population of the polypeptide loaded EVs (e.g., antibody loaded neural EVs), and optional pharmaceutical adjuvants, in a carrier, such as, for example, aqueous saline, aqueous dextrose, glycerol, or ethanol, to form a solution or suspension.

Intravenous formulations can comprise the polypeptide loaded EVs described herein (e.g., antibody loaded neural EVs), an isotonic medium and one or more substances preventing aggregation of the polypeptide loaded EVs. Example intravenous/intrathecal/intracerebrospinal fluid formulations may contain saline solutions (e.g. normal saline (NS); about 0.91% w/v of NaCl, about 300 mOsm/L) and/or dextrose 4% in 0.18% saline, and optionally 1%, 2% or 3% human serum albumin. In addition, the polypeptide loaded EVs may be disrupted to obtain the contents and the contents used in compositions according to the present invention.

For use in an oral liquid preparation, the composition may be prepared as a solution, suspension, emulsion, or syrup, being supplied either in liquid form or a dried form suitable for hydration in water or normal saline. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

In the case of intranasal, intratracheal or intrapulmonary administration, the compositions may be provided in a liquid or aerosol formulation which can be sprayed into the nose, trachea and/or lungs.

When the composition is employed in the form of a solid preparation for oral administration, the preparation may be a tablet, granule, powder, capsule or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

The pharmaceutical compositions provided herein (e.g., pharmaceutical compositions comprising the polypeptide loaded EVs) may be administered once to the subject or, alternatively, multiple administrations may be performed over a period of time. For example, two, three, four, five, or more administrations may be given to the subject during one treatment, or over a set period of time. In some instances, six, eight, ten, 12, 15 or 20 or more administrations may be given to the subject during one treatment or over a period of time as a treatment regimen. In other instances, administrations may be given as needed, e.g., for as long as symptoms associated with a neurological disorder persist. In some embodiments, repeated administrations may be indicated for the remainder of the subject's life. Exemplary dosing schedules include administration of a pharmaceutical composition comprising the polypeptide loaded EVs once per day, once every two days, once every three days, once per week, once every two weeks, once every month, once every two months, once every three months, six months, 12 months, or longer.

In some aspects, the invention provides a pharmaceutical composition comprising the polypeptide loaded EVs (e.g., antibody loaded neural EVs), which further comprises one more additional therapeutic agents.

In some embodiments, the EVs can comprise one or more inhibitory nucleic acids. For example, in some embodiments, the EVs can comprise one or more inhibitory nucleic acids selected from short interfering RNAs (siRNAs), short hairpin RNAs (shRNA), micro RNAs (miRNAs), antisense oligonucleotides (ASOs), and double-strand RNAs (dsRNA).

In some embodiments, the EVs can comprise one more neurotrophic agents. In some embodiments, the EVs can comprise one or more agents selected from leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

Pharmaceutical compositions comprising the polypeptide loaded EVs as described herein may be administered to a subject as a monotherapy (a single agent) or in a combination therapy where the subject is administered a pharmaceutical composition comprising the polypeptide loaded EVs in combination with one or more additional agents. A pharmaceutical composition comprising the polypeptide loaded EVs, and one or more additional agents, can be administrated to a subject simultaneously, sequentially or temporally.

In one embodiment, the disclosure provides a pharmaceutical composition comprising the polypeptide loaded EVs as described herein (e.g., a pharmaceutical composition comprising e.g., antibody loaded neural EVs) for use in treating a subject having a neurological disorder, for example, Alzheimer's Disease, Huntington's Disease, Parkinson's Disease, neurological cancer (e.g., glioblastoma or neuroblastoma), neuromyelitis optica, multiple sclerosis, migraine, chronic inflammatory demyelinating polyneuropathy (CIDP), or multifocal motor neuropathy. In another embodiment, the disclosure provides a pharmaceutical composition comprising the polypeptide loaded EVs as described herein (e.g., antibody-loaded EVs, including antibody-EV conjugates and EVs lumenally loaded with antibody) for use in the manufacture of a medicament for treating a subject having a neurological disorder, for example, Alzheimer's Disease, Huntington's Disease, Parkinson's Disease, neurological cancer (e.g., glioblastoma or neuroblastoma), neuromyelitis optica, multiple sclerosis, migraine, chronic inflammatory demyelinating polyneuropathy (CIDP), or multifocal motor neuropathy.

In some instances, pharmaceutical formulations may comprise about 50 ng of the polypeptide loaded EVs/ml fluid medium, or more. Exemplary pharmaceutical formulations can comprise about 100 ng, 200 ng, 300 ng, 400 ng, 500 ng, 600 ng, 700 ng, 800 ng, 900 ng, 1.0 μg, 1.5 μg, 2.0 μg, 2.5 μg, 3.0 μg, 5.0 μg, 10.0, 15.0 μg, 20.0 μg, 100 μg, or more polypeptide loaded EVs/ml fluid medium.

In other embodiments, pharmaceutical formulations may comprise about 0.1 μg polypeptide loaded EVs/ml medium, about 0.2 μg polypeptide loaded EVs/ml intravenous medium, about 0.3 μg polypeptide loaded EVs/ml intravenous medium, about 0.4 μg polypeptide loaded EVs/ml intravenous medium, about 0.5 μg polypeptide loaded EVs/ml intravenous medium, about 0.6 μg polypeptide loaded EVs/ml intravenous medium, about 0.7 μg polypeptide loaded EVs/ml intravenous medium, about 0.8 μg polypeptide loaded EVs/ml intravenous medium, about 0.9 μg polypeptide loaded EVs/ml intravenous medium, about 1.0 μg polypeptide loaded EVs/ml intravenous medium, about 1.5 μg polypeptide loaded EVs/ml intravenous medium, about 2.0 μg polypeptide loaded EVs/ml intravenous medium, about 2.5 μg polypeptide loaded EVs/ml intravenous medium, such as at least e.g. about 3.0 μg polypeptide loaded EVs/ml intravenous medium, such as e.g. at least about 5.0 μg polypeptide loaded EVs/ml intravenous medium, about 10.0 μg polypeptide loaded EVs/ml intravenous medium, 15.0 μg polypeptide loaded EVs/ml intravenous medium or about 20.0 μg or more polypeptide loaded EVs/ml intravenous medium.

In some instances, administering a composition includes 1×10⁸ or more polypeptide loaded EVs per kilogram in a single dose. In other instances, administering polypeptide loaded EVs composition includes a dosage of 1×10⁸, 1×10⁸ to 1×10⁹, 1×10⁹ to 1×10¹⁰, 1×10¹⁰ to 1×10¹¹, 1×10¹¹ to 1×10¹², 1×10¹² or more polypeptide loaded EVs per kilogram. In some instances, a single dose is administered multiple times to the subject. In certain other instances, the multiple administrations to the subject include two or more of intravenous, intracerebrospinal, intravenous infusion, and injection.

In some instances, the pharmaceutical composition is in a dosage form comprising at least 1 mg of polypeptide loaded EVs, at least 5 mg of polypeptide loaded EVs, at least 10 mg of polypeptide loaded EVs, at least 20 mg of polypeptide loaded EVs, at least 25 mg of polypeptide loaded EVs, at least 50 mg of polypeptide loaded EVs, at least 60 mg of polypeptide loaded EVs, at least 75 mg of polypeptide loaded EVs, at least 100 mg of polypeptide loaded EVs, at least 150 mg of polypeptide loaded EVs, at least 200 mg of polypeptide loaded EVs, at least 250 mg of polypeptide loaded EVs, at least 300 mg of polypeptide loaded EVs, about 350 mg of polypeptide loaded EVs, about 400 mg of polypeptide loaded EVs, about 500 mg of polypeptide loaded EVs, about 750 mg of polypeptide loaded EVs, about 1 g (1,000 mg) or more of polypeptide loaded EVs, alone or in combination with a therapeutically effective amount of at least one additional agent. In some embodiments, the pharmaceutical composition comprises between about 10 mg to about 750 mg, about 25 mg to about 650 mg, or between about 30 mg to about 500 mg, or about 35 mg to about 450 mg, most often about 50 to about 500 mg of polypeptide loaded EVs.

A therapeutically effective amount of a pharmaceutical composition comprising polypeptide loaded EVs, e.g., comprising antibody-loaded EVs, including antibody-EV conjugates and EVs lumenally loaded with antibody is an amount sufficient to treat or ameliorate one or more symptoms of the condition being treated (e.g., a neurological disorder, for example, Alzheimer's Disease, Huntington's Disease, Parkinson's Disease, or neurological cancer, e.g., glioblastoma), while not exceeding an amount which may cause significant adverse effects. Dosages that are therapeutically effective can depend on many factors including the nature of the condition to be treated as well as the particular individual being treated.

### D. Methods of Treatment

In some aspects, the invention provides a method of treating a subject using compositions comprising the polypeptide loaded EVs (e.g., antibody-loaded EVs, including antibody-EV conjugates and EVs lumenally loaded with antibody). Any of the compositions or pharmaceutical compositions comprising the polypeptide loaded EVs described herein are suitable for use in any of the methods provided herein. In exemplary embodiments, the EVs the polypeptide loaded EVs are derived from neural cells, e.g., neural progenitor cells, neurons, or astrocytes. In other exemplary embodiments, the EVs of the polypeptide loaded EVs are produced synthetically, and contain one or more markers characteristic of neural EVs, e.g., one or more proteins or nucleic acids present in neural EVs that are absent in EVs derived from MSCs.

In some aspects, the invention provides a method of treating (e.g., curing, suppressing, ameliorating associated symptoms of, delaying or preventing the progression of, delaying or preventing onset of, or preventing recurrence or relapse of) a neurological disorder, for example, Alzheimer's Disease, Huntington's Disease, Parkinson's Disease, neurological cancer (e.g., glioblastoma or neuroblastoma), neuromyelitis optica, multiple sclerosis, migraine, chronic inflammatory demyelinating polyneuropathy (CIDP), or multifocal motor neuropathy in a subject, comprising administering to the subject a composition comprising the polypeptide loaded EVs as described herein, in an amount sufficient to treat the disease or disorder in the subject. The amount sufficient to treat the disease or disorder is preferably an effective amount, e.g., a therapeutically effective amount, as provided herein.

Alteration of symptoms as a result of treatment can be measured relative to any suitable control. For example, alteration of symptoms can be measured relative to the frequency, severity, or duration, or number of symptoms experienced by the same subject prior to initiating treatment. In other embodiments, alteration of symptoms can be measured relative to the frequency, severity, duration, or number of symptoms experienced by a different subject, or group of subjects, with like symptoms who do not receive the treatment, i.e., who do not receive a composition comprising Ab-EVs. In some embodiments, the degree of improvement is at least 5%, i.e., at least 5%, 10%, 20%, 30%, 40%, 50%, 60%, 70%, 80%, 90%, 95%, 99% or more, as determined relative to a suitable control.

In some embodiments, a composition comprising the polypeptide loaded EVs is administered to a subject as a single dose. In some embodiments, a composition comprising Ab-EVs is administered in multiple doses. For example, the composition can be administered, in some embodiments, once every day, once every 2 days, once every 3 days, once every 4 days, once every 5 days, once every 6 days, once every 7 days, once every 2 weeks, once every 3 weeks, once every 4 weeks, once every 8 weeks, or once every 12 weeks.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### EXAMPLES

The Examples below are merely illustrative, and are not intended to limit the scope of the disclosure provided herein in any way.

### Example 1: Preparation of Neural Progenitor Extracellular Vesicles Surface-Labeled with Antibody

This example describes the conjugation of an antibody (Ab) to extracellular vesicles (EVs) derived from neural progenitor cells, using click chemistry.

A purified population of EVs was obtained from neural progenitor cells in accordance with the methods provided in U.S. Patent Publication No. US2018/0327714A1, the entire contents of which are incorporated by reference herein. Briefly, human neural progenitor cells were cultured as described in U.S. Patent Publication No. US2018/0327714A. Cell medium was collected from confluent cultures of neural progenitor cells 24 hours post media change, and frozen at −20° C. Medium was thawed at 4° C. overnight and filtered through a 0.22 μm filter unit prior to extracellular vesicle (EV) purification. Extracellular vesicles were purified from filtered cell culture medium using tangential flow filtration.

A goat anti-rabbit IgG antibody labeled with AlexaFluor Plus 647 was selected for proof of concept experiments due to its expected lack of reactivity and immunogenicity in a murine model. The antibody (Ab) was modified to include azide groups using the SITECLICK™ Antibody Azido Modification method (see ThermoFisher Catalog No. S20026). Manufacturer's recommendations were followed.

Separately, EVs were modified to display sDIBO via an SDP ester group which targets amine groups on the surface of EVs. sDIBO (CLICK-IT™ SDP Ester sDIBO Alkyne, ThermoFisher Catalog No. C20025) was reacted with EVs for 2 hours at room temperature at a ratio of 5.95e5 molecules of sDIBO per EV. This number was chosen as it was ˜10× the number of Ab molecules added at a later step.

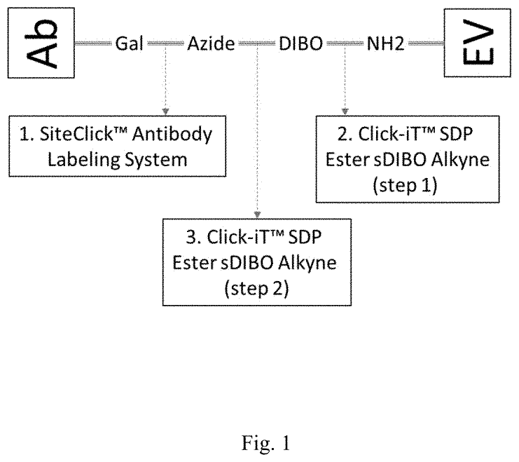

A dose-escalation experiment of free, unconjugated antibody indicated that injections of about 8×10¹³ Ab in a 100 μL dose were needed to qualitatively detect high signal in the liver, and thus was chosen as the dose to cross link to EVs. Azido-Ab were mixed with sDIBO-EV at a ratio of 5.95e4 Ab per EV. A copperless click chemistry reaction then conjugated the antibody to EV when mixed (FIG. 1).

### Example 2: In Vivo Administration of EV-Ab Conjugates

This example describes in vivo administration of the extracellular vesicle-antibody (EV-Ab) conjugate in mice. Animals were injected intravenously with the EV-Ab mixture, Ab only and PBS (n=1/condition) after overnight incubation. After about 2 hours, animals were sacrificed and brains were preserved. Tissues were fixed, cryosectioned, and in some cases treated with TrueBlack Lipofuscion Autofluorescence Quencher (Biotium) to reduce background. Sections were stained with an anti-goat secondary antibody prior to imaging.

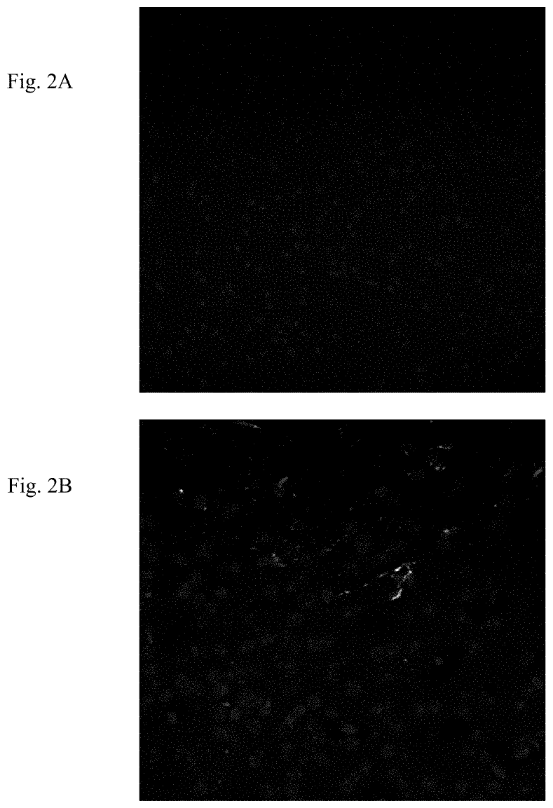

Stained and unstained sections were imaged on a Zeiss LSM 710 confocal microscope. Images were taken at multiple wavelengths to distinguish background from signal including, in some cases, taking lambda images with linear unmixing using the Zeiss Zen 2012 Blue software. Initially, randomly selected frames from brain sections of PBS-treated animals were imaged in order to get a feel for signal-to-noise. Once the untreated brain sections were imaged and analyzed, animals treated with Ab-EV mixtures were imaged as hemisphere slices in automated tiled z-stacks. Upon imaging the entire section at multiple wavelengths at low magnification, sections were examined manually using ImageJ software to zoom and scan. One hemisphere of an Ab-treated animal was scanned and analyzed, as well. After analysis, one Ab-EV hemisphere (FIG. 2B) had patterns of signal unlike either the PBS-treated or Ab only (FIG. 2A) treated brain slices. Both Ab only and PBS-treated mice showed high intensity punctate fluorescence that varied in its appearance by region, indicative of autofluorescence. In regions without punctate fluorescence, signal was completely quenched by TrueBlack Lipofuscion Autofluorescence Quencher (Biotium). The Ab-EV hemisphere showed similar autofluorescence in similar regions to PBS and Ab only-treated brain slices. However, in regions that were quenched in Ab only and PBS treated brains, Ab-EV displayed patterns of signal accumulation at high intensity, indicative of signal accumulation in the shape of a cell, at high intensity (FIG. 2B), demonstrating delivery of the Ab-EV conjugate to brain tissue.

### Example 3: Luminal Loading of Extracellular Vesicles

This example describes the loading of a polypeptide (e.g., an antibody or luciferase protein) into the lumen of extracellular vesicles derived from neural progenitor cells through EV membrane permeabilization.

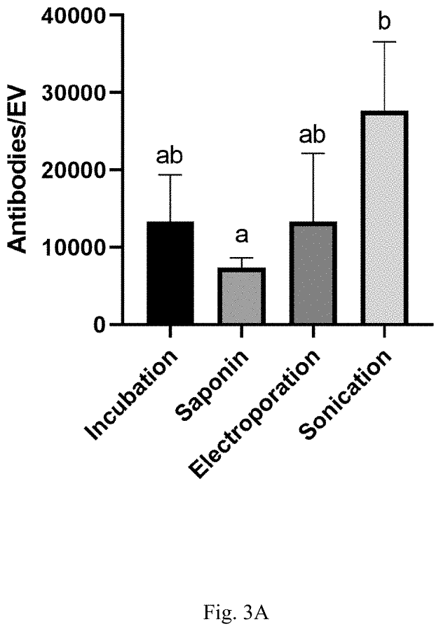

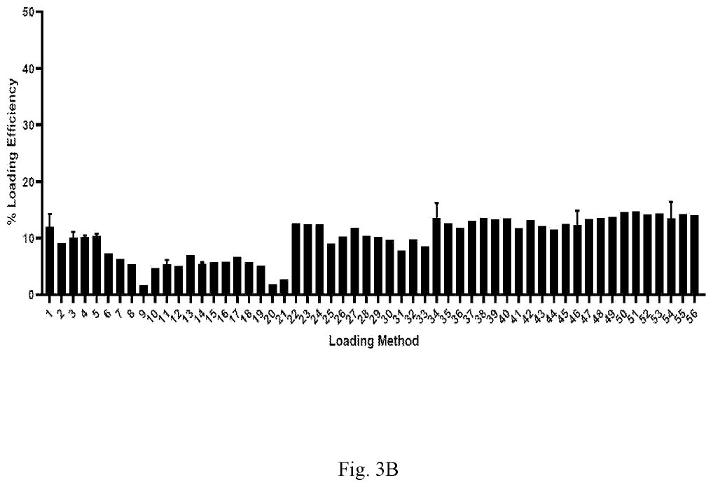

Antibody was loaded into neural progenitor-derived EVs by saponin permeabilization (0.2% w/w), incubation, electroporation, or sonication. Saponin permeabilization offers detergent-based loading and was combined with shaking (500 rpm). Incubation was performed at room temperature with shaking (1000 rpm). Electroporation was performed using a Neon Transfection system at 500V, 1 ms width, and 12 pulses. Lastly, sonication was accomplished using a Fisherbrand Model 505 Sonicator (500 W, 20 kHz) using 20% amplitude, 12 cycles, 4 seconds on and 8 seconds off. After testing these different loading procedures, antibody loading was achieved into the lumen of exosomes with upwards of 25,000 antibody molecules per exosome (EV). FIG. 3A.

Luciferase protein (62 kDa) was loaded into the neural progenitor EVs by saponin permeabilization, incubation, or sonication. Saponin permeabilization offers detergent-based loading and was combined with sonication prior to adding protein. Incubation was performed at room temperature with shaking (1000 rpm). Lastly, sonication was accomplished using a Fisherbrand Model 505 Sonicator (500 W, 20 kHz). Several permutations were tested that changed amplitude, pulse time, and number of cycles (described in Table 12 below). All sonication protocols were performed using a cup horn accessary, as direct probe sonication destroyed luciferase protein function. After testing over fifty loading procedures, upwards of 15% loading efficiency of luciferase protein was achieved. Loading efficiency is described in FIG. 3B, for each condition described in Table 12.

TABLE 12
Conditions tested for polypeptide luminal loading into EVs

| Condition # | Loading condition description |
|---|---|
| 1 | Saponin (0.2%) sonicated 20% Amp, 10 min |
| 2 | Saponin (0.2%) sonicated 40% Amp, 10 min |
| 3 | 20% Amp, 12 cycles, 4 sec on/8 sec off |
| 4 | 40% Amp, 12 cycles, 4 sec on/8 sec off |
| 5 | 60% Amp, 12 cycles, 4 sec on/8 sec off |
| 6 | 20% Amp, 36 cycles, 4 sec on/8 sec off |
| 7 | 40% Amp, 36 cycles, 4 sec on/8 sec off |
| 8 | 60% Amp, 36 cycles, 4 sec on/8 sec off |
| 9 | Incubation 18 h at room temp, shaking |
| 10 | 20% Amp, 24 cycles, 4 sec on/8 sec off |
| 11 | 40% Amp, 24 cycles, 4 sec on/8 sec off |
| 12 | 60% Amp, 24 cycles, 4 sec on/8 sec off |
| 13 | 20% Amp, 12 cycles, 4 sec on/2 sec off |
| 14 | 40% Amp, 12 cycles, 4 sec on/2 sec off |
| 15 | 60% Amp, 12 cycles, 4 sec on/2 sec off |
| 16 | 20% Amp, 24 cycles, 4 sec on/2 sec off |
| 17 | 40% Amp, 24 cycles, 4 sec on/2 sec off |
| 18 | 60% Amp, 24 cycles, 4 sec on/2 sec off |
| 19 | 20% Amp, 36 cycles, 4 sec on/2 sec off |
| 20 | 40% Amp, 36 cycles, 4 sec on/2 sec off |
| 21 | 60% Amp, 36 cycles, 4 sec on/2 sec off |
| 22 | 20% Amp, 12 cycles, 2 sec on/4 sec off |
| 23 | 40% Amp, 12 cycles, 2 sec on/4 sec off |
| 24 | 60% Amp, 12 cycles, 2 sec on/4 sec off |
| 25 | 20% Amp, 24 cycles, 2 sec on/4 sec off |
| 26 | 40% Amp, 24 cycles, 2 sec on/4 sec off |
| 27 | 60% Amp, 24 cycles, 2 sec on/4 sec off |
| 28 | 20% Amp, 36 cycles, 2 sec on/4 sec off |
| 29 | 40% Amp, 36 cycles, 2 sec on/4 sec off |
| 30 | 60% Amp, 36 cycles, 2 sec on/4 sec off |
| 31 | 20% Amp, constant 5 min |
| 32 | 20% Amp, 6 cycles, 4 sec on/8 sec off |
| 33 | 40% Amp, 6 cycles, 4 sec on/8 sec off |
| 34 | 60% Amp, 6 cycles, 4 sec on/8 sec off |
| 35 | 20% Amp, 6 cycles, 4 sec on/2 sec off |
| 36 | 40% Amp, 6 cycles, 4 sec on/2 sec off |
| 37 | 60% Amp, 6 cycles, 4 sec on/2 sec off |
| 38 | 20% Amp, 6 cycles, 2 sec on/4 sec off |
| 39 | 40% Amp, 6 cycles, 2 sec on/4 sec off |
| 40 | 60% Amp, 6 cycles, 2 sec on/4 sec off |
| 41 | Saponin (0.2%) sonicated 20% Amp, 5 min |
| 42 | 80% Amp, 6 cycles, 4 sec on/8 sec off |
| 43 | 80% Amp, 6 cycles, 4 sec on/2 sec off |
| 44 | 80% Amp, 6 cycles, 2 sec on/4 sec off |
| 45 | 80% Amp, 12 cycles, 2 sec on/4 sec off |
| 46 | 100% Amp, 6 cycles, 4 sec on/8 sec off |
| 47 | 100% Amp, 6 cycles, 4 sec on/2 sec off |
| 48 | 100% Amp, 6 cycles, 2 sec on/4 sec off |
| 49 | 60% Amp, 6 cycles, 4 sec on/8 sec off, ice 2 min, repeat |
| | once |
| 50 | 60% Amp, 6 cycles, 4 sec on/2 sec off, ice 2 min, repeat |
| | once |
| 51 | 60% Amp, 6 cycles, 2 sec on/4 sec off, ice 2 min, repeat |
| | once |
| 52 | 80% Amp, 6 cycles, 4 sec on/8 sec off, ice 2 min, repeat |
| | once |
| 53 | 80% Amp, 6 cycles, 4 sec on/2 sec off, ice 2 min, repeat |
| | once |
| 54 | 80% Amp, 6 cycles, 2 sec on/4 sec off, ice 2 min, repeat |
| | once |
| 55 | 100% Amp, 6 cycles, 4 sec on/8 sec off, ice 2 min, repeat |
| | once |
| 56 | 100% Amp, 6 cycles, 2 sec on/4 sec off, ice 2 min, repeat |
| | once |

Amp = amplitude

### INCORPORATION BY REFERENCE

The contents of all references, patents, pending patent applications, and publications cited throughout this application are hereby expressly incorporated by reference herein in their entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of delivering an antibody, or antigen binding portion thereof, to the central nervous system (CNS) of a subject, comprising administering to the subject:
(i) a conjugate comprising an antibody or antigen binding portion thereof and an extracellular vesicle (EV) derived from a neural cell, wherein the antibody or antigen binding portion thereof is conjugated to the surface of the extracellular vesicle by way of a click linker; or
(ii) an EV that comprises the antibody or antigen binding portion thereof in the lumen of the EV, wherein the EV is derived from a neural cell.

2. The method of claim 1, wherein the conjugate or the EV is administered intravenously, intranasally, intracranially, or intrathecally.

3.-5. (canceled)

6. The method of claim 1, wherein the neural cell is a neural progenitor cell.

7. The method of claim 6, wherein the neural progenitor cell is derived from a human pluripotent cell.

8. The method of claim 7, wherein the human pluripotent cell is a human embryonic stem cell or a human induced pluripotent stem cell.

9. (canceled)

10. The method of claim 1, wherein the antibody, or antigen binding portion thereof, is an IgG or an antibody fragment, wherein the antibody fragment is selected from the group consisting of a Fab, a F(ab′)2, an scFv, a tandem scFv, a diabody, a minibody, and a single domain antibody.

11. (canceled)

12. The method of claim 1, wherein the antibody or antigen binding portion thereof is a humanized or fully human antibody or antigen binding portion thereof.

13. (canceled)

14. The method of claim 1, wherein the click linker is formed from reaction between: an azide click reagent and an alkyne click reagent; azide and dibenzocyclooctyne (DBCO); tetrazine and transcyclooctene; and/or tetrazine and norbornene.

15.-17. (canceled)

18. The method of claim 1, wherein the antibody or antibody binding portion thereof is delivered to the brain or the spinal cord of the subject.

19. (canceled)

20. The method of claim 1, wherein the EV further comprises an exogenous nucleic acid, an exogenous protein, an exogenous siRNA, an antisense nucleic acid, and/or a small molecule.

21.-22. (canceled)

23. A composition comprising an antibody-EV (Ab-EV) conjugate, the conjugate comprising an antibody, or antigen binding portion thereof, and an extracellular vesicle (EV) derived from a neural cell, wherein the antibody, or antigen binding portion thereof, is conjugated to the EV surface by a click linker.

24. The composition of claim 23, wherein the neural cell is a neural progenitor cell.

25. The composition of claim 24, wherein the neural progenitor cell is derived from a human pluripotent cell.

26. The composition of claim 25, wherein the human pluripotent cell is a human embryonic cell or an induced pluripotent cell.

27. The composition of claim 23, wherein the click linker is formed from reaction between: azide and dibenzocyclooctyne; tetrazine and transcyclooctene; tetrazein and norbornene; azide and alyne; azide (strain-promoted) and alkyne; azide (strain-promoted) and nitrone; alkene and azide; alkene and tetrazine; and/or alkene and tetrazole.

28. The composition of claim 23, wherein the antibody, or antigen binding portion thereof, is an IgG or an antibody fragment, wherein the antibody fragment is selected from the group consisting of a Fab, a F(ab′)2, an scFv, a tandem scFv, a diabody, a minibody, and a single domain antibody.

29. (canceled)

30. The composition of claim 23, wherein the antibody, or antigen binding portion thereof is a humanized or fully human antibody or antigen binding portion thereof.

31. The composition of claim 23, wherein the antibody, or antigen binding portion thereof, is any one of more of solanezumab, aducanumab, nivolumab, bevacizumab, ocrelizumab, natalizumab, dinutuximab, gantenerumab, lecanemab, or ublituximab.

32. A method of loading an antibody, or antigen binding portion thereof, into the lumen of an extracellular vesicle (EV), comprising: i) treating the EV with saponin to permeabilize the EV membrane; ii) sonicating the treated EV; and iii) adding the antibody, or antigen-binding portion thereof, to the EV, thereby loading an antibody, or antigen-binding portion thereof, into the lumen of the EV.

33. The method of claim 32, wherein the EV is treated with about 0.05% to 0.3% saponin.

34.-48. (canceled)
